(19) United States
(12) Patent Application Publication
(10) Pub. No.: US 2002/0110926 A1
(43) Pub. Date: Aug. 15, 2002

(54) Emulator device

(75) Inventors: Anne R. Kopf-Sill, Portola Valley, CA (US); Andrea W. Chow, Los Altos, CA (US); Michael Spaid, Sunnyvale, CA (US); J. Wallace Parce, Palo Alto, CA (US)

Correspondence Address: CALIPER TECHNOLOGIES CORP, 605 FAIRCHILD DRIVE, MOUNTAIN VIEW, CA, 94043, US

(73) Assignee: Caliper Technologies Corp., Mountain View, CA

(21) Appl. No.: 10/044,825

(22) Filed: Jan. 11, 2002

Related U.S. Application Data
(60) Provisional application No. 60/262,010, filed on Jan. 16, 2001.

(51) Int. Cl. C12Q 1/68; C12M 1/34; G01N 33/561

(52) U.S. Cl. 436/516; 435/287.2

(57) ABSTRACT

Non-sipper microfluidic devices, e.g., planar devices that do not comprise an external capillary, are used to emulate or simulate the fluid flow profile of a device having an external capillary, e.g., a microfluidic sipper device. Samples are typically flowed through a sipper device, e.g., in sample plugs. To emulate fluid flow in a sipper device, emulator devices create sample plugs and flow them through the channels of a planar device.

### CROSS-REFERENCE TO RELATED APPLICATION

This application claims the benefit of and priority to U.S. Ser. No. 60/262,010, filed on Jan. 16, 2001, the disclosure of which is incorporated by reference in its entirety.

### BACKGROUND OF THE INVENTION

The development of microfluidic technologies by the inventors and their co-workers has provided a fundamental shift in how artificial biological and chemical processes are performed. In particular, the inventors and their co-workers have provided microfluidic systems that dramatically increase throughput for biological and chemical methods, as well as greatly reducing reagent costs for the methods. In these microfluidic systems, small volumes of fluid are moved through microchannels, e.g., by electrokinetic or pressure-based mechanisms. Fluids can be mixed and/or reacted, and the results of the experiments determined by monitoring a detectable signal from products of the experiments.

Complete integrated systems with fluid handling, signal detection, sample storage and sample accessing are available. For example, Parce et al. “High Throughput Screening Assay Systems in Microscale Fluidic Devices” WO 98/00231 and Knapp et al. “Closed Loop Biochemical Analyzers” (WO 98/45481; PCT/US98/06723) provide pioneering technology for the integration of microfluidics and sample selection and manipulation. For example, in WO 98/45481, microfluidic apparatus, methods and integrated systems are provided for performing a large number of iterative, successive, or parallel fluid manipulations. High throughput applications are described in WO 98/00231, which provides methods of sampling a plurality of compounds and sequentially or simultaneously analyzing them in a high throughput manner.

Methods of designing and optimizing assays and syntheses for use in a high throughput system are, accordingly, desirable, particularly those that take advantage of low cost microfluidic systems. The present invention provides these and other features by providing new microscale devices and methods as well as many other features that will be apparent upon complete review of the following disclosure.

### SUMMARY OF THE INVENTION

The present invention provides methods of optimizing microfluidic assays, e.g., high throughput assays. For example, high throughput microfluidic assays are optionally performed using a microfluidic device with an external capillary, which is used to sip a series of sample plugs into a microfluidic device. However, before performing a particular assay in a high throughput manner, the assay is typically designed and optimized in a relatively lower throughput manner, e.g., using a lower cost device that does not comprise an external capillary, e.g., a non-sipper or planar device. The present invention provides methods and devices for emulating the discrete sipping of sample plugs on non-sipper devices, e.g., planar devices. These methods and devices allow assays to be optimized using a planar device, e.g., for eventual performance in a high throughput manner using sipper devices.

In one aspect, the invention provides methods of flowing fluid in a non-sipper microfluidic device. The methods comprise flowing fluid through a non-sipper microfluidic device, e.g., a planar device, to emulate a fluid flow profile in a microfluidic device comprising an external capillary, e.g., a sipper device. The fluid flow profile of the sipper device results from flowing one or more sample from an external source into a microfluidic device, e.g., sipping discrete sample plugs from a microwell plate into a microfluidic channel through an external capillary.

Fluid flow through a non-sipper microfluidic device, e.g., to emulate a sipper device, typically comprises creating one or more sample plug and one or more buffer plug in the non-sipper microfluidic device. The sample plugs and buffer plugs emulate the flow of fluid from an external source into a microfluidic device, e.g., a sipper device, via an external capillary.

In one embodiment, creating the sample plugs and buffer plugs in the non-sipper device comprises loading a sample from a first source, e.g., an internal source, into a channel of the non-sipper microfluidic device and loading a buffer, e.g., from a second source. The method further comprises applying pressure, e.g., continuously, to the sample in the channel, to create a sample plug and transport it through the channel, and applying pressure, e.g., continuously, to the buffer in the channel to create a buffer plug and transport it through the channel.

The sample and buffer are alternately loaded and repeated to create multiple sample plugs and buffer plugs in the channel. For example, a portion of sample is loaded, e.g., from an internal reservoir, followed by a portion of buffer, e.g., from a second internal reservoir, while simultaneously applying pressure to the sample and the buffer in the channel to transport the plugs through the channel. In some embodiments, different pressures are applied to the sample and buffer, e.g., at the internal reservoirs that comprise the sample material and buffer material.

In one embodiment, samples and buffers are loaded, e.g., from internal sources or reservoirs, into a microfluidic channel of a non-sipper microfluidic device by applying electrokinetic gradients. For example, a first electrokinetic gradient is applied between a sample source and a waste reservoir, e.g., an internal reservoir, thereby loading a sample into the microfluidic channel, and a second electrokinetic gradient is applied between a buffer source and the waste reservoir, thereby loading the buffer into the microfluidic channel. The two electrokinetic gradients, e.g., of equal or unequal magnitude, are typically applied in an alternate manner to load sample and then buffer and then sample and so on. Pressure is simultaneously applied to the microfluidic channel to transport fluid in the channel, thereby creating sample and buffer plugs and transporting them through the channel. In other embodiments, the samples and buffers are loaded into the microfluidic channel using pressure, e.g., by alternately applying pressure to the sample reservoir and to the buffer reservoir.

In another embodiment, flowing fluid through a non-sipper microfluidic device, e.g., to emulate a sipper device, comprises flowing a sample, e.g., from an internal source, into a non-sipper main channel via a capillary emulator channel, which emulates or simulates an external capillary. The sample is then typically flowed through the non-sipper main channel and one or more reagent, e.g., from a second internal source, is optionally flowed into the non-sipper main channel, e.g., via a non-sipper side channel, to react with the sample. The non-sipper main channel and non-sipper side channel typically simulate the main side channels in a sipper device, eg., a sipper main channel and a sipper side channel.

Simulation of a sipper channel by a non-sipper channel typically comprises providing the non-sipper channel or channels to have substantially the same hydrodynamic resistance as an equivalent channel in a microfluidic device comprising an external capillary, having substantially the same length, width, and/or depth as an equivalent sipper channel, or flowing substantially the same amount reagent or sample as an equivalent sipper channel, e.g., in the same amount of time. Additionally, the non-sipper channel is optionally set up to produce equivalent dispersion as a corresponding sipper channel.

In another aspect, the present invention provides assay development devices that emulate a microfluidic sipper device. The assay development devices typically comprise a non-sipper microfluidic substrate, e.g., a planar microfluidic substrate, comprising a plurality of microscale channels. The plurality of microscale channels typically comprises a main channel and at least one capillary emulator fluidly coupled to the main channel. In addition, the devices typically comprise at least one fluid control element fluidly coupled to the main channel. Fluid control elements typically comprise one or more pressure source and/or electrokinetic controller.

The main channel of the assay device typically emulates a sipper device main channel, e.g., by having a hydrodynamic resistance, a length, a width, a depth, or a flow characteristic that is substantially equal to the sipper device main channel.

The capillary emulator typically comprises at least one microscale channel. The microscale channel typically comprises a hydrodynamic resistance, a length, a width, a depth, or a flow characteristic, that is substantially equal to a sipper capillary in the microfluidic sipper device which is being emulated.

In some embodiments, the assay development device further comprises an electrokinetic controller fluidly coupled to the capillary emulator. The capillary emulator optionally comprises a waste reservoir fluidly coupled to the main channel, a sample well fluidly coupled to the waste reservoir and to the main channel, and a buffer well fluidly coupled to the waste reservoir and to the main channel. The fluid control element optionally applies a pressure differential between the waste reservoir and the pressure source, and the electrokinetic controller, e.g., simultaneously with the pressure differential applied by the fluid control element, alternately applies an electrokinetic gradient between the sample well and the waste reservoir and between the buffer well and the waste reservoir.

In other embodiments, the capillary emulator comprises a sample well fluidly coupled to the main channel of the emulator device and a buffer well fluidly coupled to the main channel. The first fluid control element optionally applies a first pressure to the sample in the sample well, thereby flowing the sample into the main channel, and applies a second pressure to the buffer in the buffer well, thereby flowing the buffer into the main channel. A third pressure is typically applied to the sample or buffer in the main channel, e.g., to form sample and buffer plugs that are transported through the main channel, thereby emulating a microfluidic sipper device. For example, the fluid control element optionally alternates between applying the first pressure and applying the second pressure while concurrently applying the third pressure.

For example, a typical assay development device of the invention comprises a non-sipper microfluidic device and a fluid control system. The microfluidic device comprises a plurality of microscale channels, e.g., a main channel, a first reagent well fluidly coupled to the main channel, a second reagent well fluidly coupled to the main channel, and, a waste reservoir fluidly coupled to the main channel and the reagent wells. The fluid control system typically comprises a pressure source fluidly coupled to the main channel and an electrokinetic controller operably coupled to the main channel. The fluid control system applies a pressure differential between the waste reservoir and the pressure source, and alternately applies an electrokinetic gradient between the first reagent well and the waste reservoir and between the second reagent well and the waste reservoir.

In another aspect, the present invention provides a method of fabricating an assay development device. The method comprises providing a non-sipper microfluidic substrate, e.g., a planar substrate, and fabricating two or more channels therein. The two or more channels, e.g., a capillary emulator, a main channel, a side channel, a reservoir, or a combination thereof, emulate an external capillary and the main channel of a microfluidic sipper device. Emulating the external capillary and the main channel of the microfluidic sipper device typically comprises having one or more of: substantially the same hydrodynamic resistance, substantially the same width, substantially the same depth, substantially the same length, or substantially the same flow characteristics as the channel being emulated, e.g., the external capillary or the main channel of the microfluidic sipper device. Having substantially the same flow characteristics typically comprises providing substantially the same amount of fluid flow in substantially the same amount of time, e.g., as the channel being emulated.

### BRIEF DESCRIPTION OF THE FIGURES

FIG. 1: Schematic of a cross-junction dual mode injector. Panel A illustrates a sample being electrokinetically driven across a cross-junction, and pulled at full concentration toward a vacuum well. Panel B illustrates a buffer plug being injected after the sample injection shown in panel A. Panels C and D illustrate alternate channel configurations.

FIG. 2: Schematic of a steady state emulator device of the invention, e.g., a non-sipper or planar microfluidic channel system that emulates a microfluidic sipper device.

FIG. 3: Experimental data showing the washout of dye from two side channels in a microfluidic device. A time delay between the two side channels results in the characteristic stair step signal profile that is used to determine the fraction of flow delivered by each side channel. Dashed lines indicate predictions for side channel dilution,which are in excellent agreement with experimental data.

FIG. 4: Velocimetry results for a microfluidic emulator device, indicating transit time from the capillary emulator to the detection point as a function of the applied vacuum.

FIG. 5: Dual mode dye injection using an emulator device of the invention. Panel A shows the injector flowing buffer to the waste well (state 2). Panel B shows the injector during the dye injection step (state 1). Panels C and D show the device being subsequently switched back to state (2) with buffer flowing to the waste well again as in Panel A to insert a buffer plug, e.g., in a main channel.

FIG. 6: Results from a dye injection experiment using a dual mode injector emulator device of the invention at −1 psi operating vacuum.

FIG. 7: Waveform of a dual-mode injector dye pulse as a function of the pulse time, with the detector positioned immediately downstream of the injector cross junction.

FIG. 8: A schematic of a non-sipper microfluidic device of the invention.

FIG. 9: A schematic of a device comprising an external capillary.

FIG. 10: Schematic illustration of pressure loading to create sample and buffer plugs.

FIG. 11: Schematic illustration of an emulator device that functions as a dual mode injector for creating sample and buffer plugs.

### DETAILED DISCUSSION OF THE INVENTION

Microfluidic devices are often used, e.g., to perform high throughput screening of a variety of test compounds, e.g., to test a variety of enzyme inhibitors. Such high throughput assays are typically performed in a microfluidic sipper device, which typically comprises a substrate with microfluidic channels disposed therein and one or more external capillary, e.g., extending from one surface of the substrate. The external capillary is typically fluidly coupled to one or more of the microfluidic channels and is used to draw samples into the microfluidic channel system, e.g., from a microwell plate or membrane. For example, the external capillary is used to sip compounds from a microwell plate positioned beneath the microfluidic substrate. In many applications, a sample compound is sipped followed by a buffer compound to produce a series of sample plugs and buffer plugs which are transported through the microfluidic channels.

Prior to performing a high throughput assay, e.g., using sipper devices, an assay is typically designed and optimized, e.g., in a bench-top microfluidic system. High throughput assay systems using microfluidic sipper devices are available from Caliper Technologies Corp. (Mountain View, Calif.), e.g., through their Technology Access Program. The sipper devices in the high throughput system typically comprise a small glass tube or capillary inserted into the device that provides for transport of fluid, e.g., a few nanoliters at a time, into the device. Samples, e.g., large numbers of samples, are introduced into the device one after the other in this manner, typically separated or spaced by buffer solution. For example, a sipper device is used with microwell plates to introduce a series of samples from the microwell plate into the sipper device through the capillary.

Microfluidic devices for performing assays, e.g., bench-top assays, are also available commercially, e.g., such as a Personal Laboratory System such as the Agilent 2100 Bioanalyzer from Agilent Technologies (Palo Alto, Calif.). Such devices typically comprise a substrate, e.g., glass, quartz, plastic, or the like, with one or more microfluidic channels disposed therein. These devices are typically planar devices that do not comprise an external capillary. Instead accessing, e.g., microwell plates for sample introduction, samples are loaded into a device without an external capillary, e.g., using pipettes. For example, planar devices typically used for bench-top operations typically make use of internal sample reservoirs into which samples are loaded, as opposed to sipping samples from an external sample source, e.g., a microwell plate. Samples and or reagents are typically added to the reservoirs prior to or during the assay, e.g., using a pipette. Alternatively, reagents are stored, e.g., in lyophilized form, in the reservoirs.

It is often desirable to optimize a selected assay on a bench-top level before performing it in a high throughput manner. However, planar devices do not typically provide fluid flow profiles like that of high throughput systems. For example, sample and buffer plugs are not typically created when a sample is flowed, e.g., in a continuous format, from an internal reservoir instead of being sipped from an external source such as a microwell plate.

The present invention provides microfluidic emulator devices and methods of emulating fluid flow profiles, e.g., to emulate the fluid flow profile of a high throughput microfluidic sipper device in a microfluidic device that does not comprise an external capillary, e.g., a planar device. The devices of the invention are typically planar devices without an external capillary that emulate the fluid flow in a sipper device by creating sample and buffer plugs or otherwise emulating particular flow characteristics.

“Emulate” is used herein to refer to the imitation of a sipper device fluid flow profile in a planar device. The planar emulator devices of the invention emulate, e.g., imitate, equal, simulate, copy, or the like, one or more flow characteristic of a sipper device, e.g., hydrodynamic resistance, flow rate, amount of fluid flow, or the like. An emulator device permits an assay designed on a planar device to be easily transferred to a sipper device, or vice versa.

I. Non-Sipper Microfluidic Devices

One class of microfluidic devices for desk-top or bench-top assays do not comprise external capillary or sipper channels. Instead these relatively simple devices, often referred to as “planar devices,” typically use internal reservoirs as sources of reagents. Compounds are, therefore, not sipped from a microwell plate and do not form discrete plugs in the microfluidic channels. Such devices are described in detail, e.g., in U.S. Pat. No. 5,942,443, by Parce et al. “High Throughput Screening Assay Systems in Microscale Fluidic Devices” and on the Caliper Technologies Corp. website: http://www.calipertech.com.

A typical non-sipper microfluidic device or non-sipper microfluidic substrate comprises a body structure with microfluidic channels disposed therein. For example, non-sipper microfluidic devices typically comprise, e.g., a main channel, one or more side channels, and one or more internal reservoir, such as a waste reservoir. The channels are typically fluidly coupled to each other and to various reservoirs. In addition, the devices optionally comprise additional channels and/or regions, such as loading channels and/or detection regions. The non-sipper devices of the invention are typically considered to be a planar devices or substrates because they do not comprise an external capillary, e.g., a capillary or tube extending out of the body structure.

Channels in non-sipper microfluidic device are referred to herein as “non-sipper” channels. For example, a planar microfluidic device typically comprises a “non-sipper” main channel and one or more non-sipper side channels. A “non sipper main channel,” as used herein, refers to a main channel in a non-sipper or planar device, as opposed to a main channel in a device comprising an external capillary or sipper capillary. The main channel is typically used to transport, mix, and/or react samples, e.g., with various reagents. For example, an enzyme inhibition assay is optionally carried out by flowing an enzyme, an enzyme substrate, and a potential inhibitor through the main channel of a planar microfluidic device.

Side channels are typically used to flow reagents, e.g., from reservoirs, into a main channel. A “non-sipper side channel” is a side channel in a planar device. For example, a side channel is optionally used to flow an enzyme, enzyme substrate, or inhibitor sample from a reservoir into a main channel of a non-sipper microfluidic device. In the emulator devices of the invention, which are described in more detail below, the non-sipper channels mimic or simulate the sipper channels.

Materials used in the present invention include, but are not limited to, samples, reagents, buffers, and the like. For example, typical samples comprise enzyme inhibitors and typical reagents comprise enzymatic substrates. Other samples and reagents include, but are not limited to nucleic acids, e.g., DNA or RNA, polynucleotides, PCR reactants and products, enzymes, proteins, polypeptides, lipids, cells, sugars, sieving matrices, and the like. These materials are transported through the various channels of the device, e.g., using pressure based flow and/or electrokinetic flow.

Sources of materials in non-sipper devices typically comprise reservoirs, e.g., internal reservoirs disposed within the body structure of the device. Sources of materials for non-sipper devices are typically within the substrate of the device, e.g., they are not external to the device, such as in a microwell plate or other container. In some embodiments, the samples or reagents are stored or pre-disposed within the reservoirs of the devices. For example, reagents are optionally lyophilized in the reservoirs of the device prior to use. Alternatively, reagents and or samples are added into the reservoirs of the device, e.g., at the time of the assay, e.g., using a pipette.

Reservoirs, e.g., for storing, discarding, or supplying, samples, reagents, buffers, and the like, are typically included in the devices of the present invention, e.g., planar or non-sipper devices. For example, a reservoir for a binding buffer or a sample well is optionally located at one end of the side channel for introduction of the sample into the main channel. The reservoirs are the locations or wells at which samples, components, reagents and the like are added into the device for assays to take place. Introduction of these elements into the system is carried out as described below.

Pressure sources are optionally used at the reservoirs of the invention, e.g., to flow reagents from the reservoirs into the channels or to draw reagents from the channels into the reservoirs. For example a vacuum source is optionally fluidly coupled to a device, e.g., at a waste reservoir located at the end of a main channel. The vacuum source draws fluid into the main channel for mixing and/or reacting with other reagents. Additionally, the vacuum optionally draws any reaction products, or excess or unused material, into the waste reservoir to which the vacuum source is fluidly coupled, e.g., at the completion of the reaction. Alternatively, a positive pressure source is fluidly coupled to a sample well or reservoir at one end of a side channel. The pressure then forces the material into and through the main channel.

Electrokinetic, e.g., electrophoretic or electroosmotic, forces, e.g., high or low voltages or currents, are also optionally applied at reservoirs to the materials in the channels. For example, voltage gradients applied across a channel are used to move fluid down the channel, e.g., to separate various components of the material as they move through the channel at different rates.

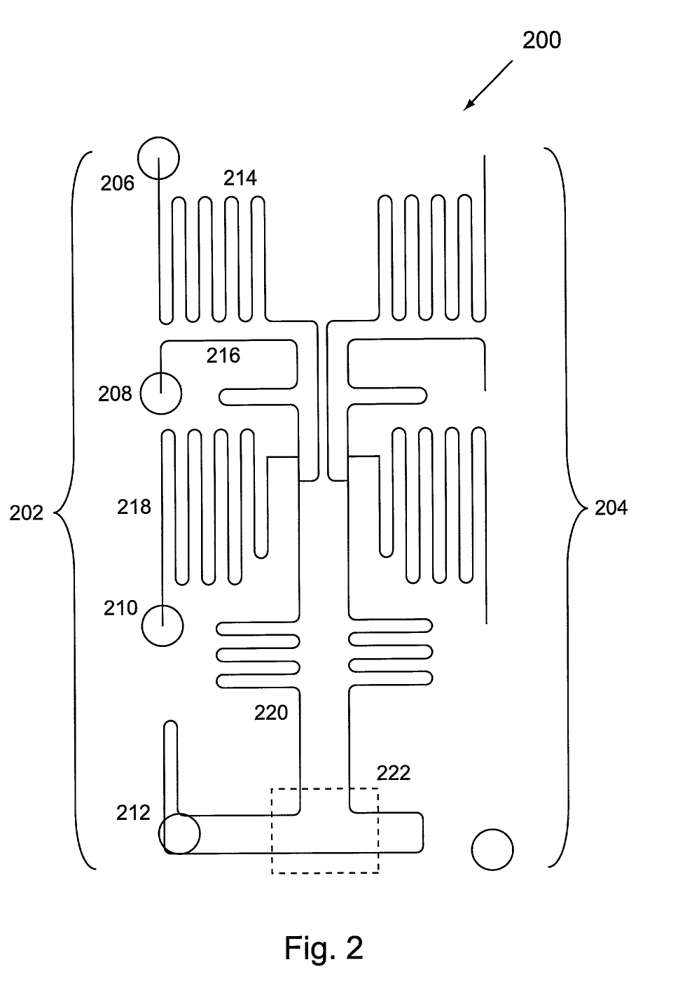

Detection regions are also included in the present devices. The detection region is optionally a subunit of a channel, such as detection region 222 in FIG. 2. Alternatively, the detection region optionally comprises a distinct channel that is fluidly coupled to a plurality of channels in the microfluidic device. For example, a channel is optionally positioned to serve as a detection channel. The detection region is optionally located anywhere along the length of a channel or region. For example, a detection region located at the most downstream point or end of a separation channel detects separated components as they exit the separation channel. In other embodiments, the detection region is optionally located at the downstream end of the device just upstream from a waste well.

The detection window or region at which a signal is monitored typically includes a transparent cover allowing visual or optical observation and detection of the assay results, e.g., observation of a colorimetric or fluorometric signal or label. Examples of suitable detectors are well known to those of skill in the art and are discussed in more detail below.

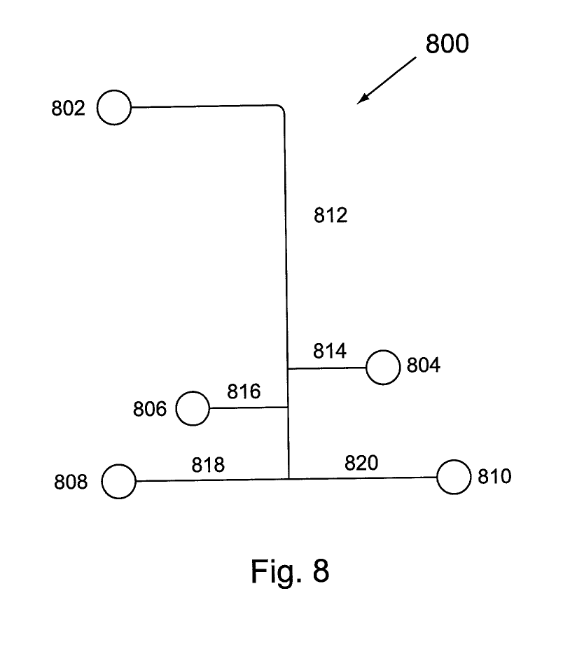

One embodiment of a non-sipper microfluidic device is illustrated in FIG. 8. As shown, the system comprises reservoirs 802, 804, 806, and 810 disposed within body structure 800, which are optionally used to introduce samples and/or reagents into the system. For example, reservoir 808 is optionally used to introduce a sample into main channel 812, e.g., through side channel 818. A sample is typically stored, e.g., a pre-disposed or dried reagent, in a reservoir, e.g., reservoir 808 for introduction into the device. Alternatively, the reagent is introduced into a reservoir, e.g., reservoir 808, using a pipette. Upon introduction into the device, the sample is typically flowed, e.g., continuously from reservoir 808 into main channel 812. The sample is then directed through main channel 812. In some embodiments, the sample is optionally diluted, e.g., with a buffer added from reservoir 810 through side channel 820. Typically, the sample is mixed with reagents in the main channel. For example, a sample in main channel 812 is optionally mixed with reagents from reservoirs 806 and 804, which are typically introduced through side channels 816 and 814. For example a DNA sample is optionally mixed with PCR reagents in main channel 812. Fluid flow in main channel 812 is typically controlled by a pressure source, e.g., a pressure source fluidly coupled to either reservoir 808 or 810, or waste reservoir 802, or electrokinetic forces applied at various reservoirs, e.g., using electrodes at reservoirs 808, 810, 806, 804, and/or 802. For example, a vacuum source is optionally coupled to waste reservoir 802. In other embodiments, an additional channel, e.g., a separation channel such as a polyacrylamide filled channel, is included in the planar device to separate various components, e.g., nucleic acids or PCR products. A detector is optionally positioned proximal to waste reservoir 802 or proximal to the downstream end of main channel 812 to detect components as they flow through the channel, e.g., at a detection window. When the assay and detection are complete, the sample components are optionally directed to waste reservoir 802 for disposal or retrieval. Any of the reservoirs, e.g., 802, 804, 806, 808, and 810 are optionally used as waste wells.

In a typical planar device, reagents and samples are mixed in a main channel as they are flowed, e.g., in a continuous stream, from a reservoir into the main channel of a device. These channels do not therefore produce discrete samples separated by spacers, e.g., buffer plugs. In addition, the channels optionally have different lengths, widths, and other flow characteristics from corresponding channels in sipper devices. Therefore, the same reaction times and/or incubation times are not typically achieved in planar devices as in sipper devices. In addition, dispersion and diffusion characteristics are different in planar devices because of the lack of discrete sample plugs. Therefore, an assay is not typically optimized for high throughput format using a planar device, e.g., in a benchtop individual research station. The present invention provides planar devices that emulate sipper devices and can therefore be used to optimize assays for high throughput format on planar benchtop systems.

The channels and devices described above are examples of possible channel systems. However, various configurations and dimensions are possible to accommodate the fluid flow profiles described herein. In fact, a variety of microscale systems are optionally adapted to the present invention, e.g., in a non-sipper device format, a sipper device format, or an emulator device format. Microfluidic devices, e.g., non-sipper and sipper devices, which can be adapted to the present invention using the fluid flow techniques described herein are described in various PCT applications and issued U.S. Patents by the inventors and their coworkers, including U.S. Pat. No. 5,699,157 (J. Wallace Parce) issued Dec. 16, 1997, U.S. Pat. No. 5,779,868 (J. Wallace Parce et al.) issued Jul. 14, 1998, U.S. Pat. No. 5,800,690 (Calvin Y. H. Chow et al.) issued Sep. 1, 1998, U.S. Pat. No. 5,842,787 (Anne R. Kopf-Sill et al.) issued Dec. 1, 1998, U.S. Pat. No. 5,852,495 (J. Wallace Parce) issued Dec. 22, 1998, U.S. Pat. No. 5,869,004 (J. Wallace Parce et al.) issued Feb. 9, 1999, U.S. Pat. No. 5,876,675 (Colin B. Kennedy) issued Mar. 2, 1999, U.S. Pat. No. 5,880,071 (J. Wallace Parce et al.) issued Mar. 9, 1999, U.S. Pat. No. 5,882,465 (Richard J. McReynolds) issued Mar. 16, 1999, U.S. Pat. No. 5,885,470 (J. Wallace Parce et al.) issued Mar. 23, 1999, U.S. Pat. No. 5,942,443 (J. Wallace Parce et al.) issued Aug. 24, 1999, U.S. Pat. No. 5,948,227 (Robert S. Dubrow) issued Sep. 7, 1999, U.S. Pat. No. 5,955,028 (Calvin Y. H. Chow) issued Sep. 21, 1999, U.S. Pat. No. 5,957,579 (Anne R. Kopf-Sill et al.) issued Sep. 28, 1999, U.S. Pat. No. 5,958,203 (J. Wallace Parce et al.) issued Sep. 28, 1999, U.S. Pat. No. 5,958,694 (Theo T. Nikiforov) issued Sep. 28, 1999, and U.S. Pat. No. 5,959,291 (Morten J. Jensen) issued Sep. 28, 1999; and published PCT applications, such as, WO 98/00231, WO 98/00705, WO 98/00707, WO 98/02728, WO 98/05424, WO 98/22811, WO 98/45481, WO 98/45929, WO 98/46438, and WO 98/49548, WO 98/55852, WO 98/56505, WO 98/56956, WO 99/00649, WO 99/10735, WO 99/12016, WO 99/16162, WO 99/19056, WO 99/19516, WO 99/29497, WO 99/31495, WO 99/34205, WO 99/43432, and WO 99/44217.

For example, pioneering technology providing cell based microscale assays, e.g., in planar and sipper format, are set forth in U.S. Pat. No. 5,942,443, by Parce et al. “High Throughput Screening Assay Systems in Microscale Fluidic Devices” and, e.g., in No. 60/128,643 filed Apr. 4, 1999 and Ser. No. 09/510,626 filed Feb. 22, 2000, both entitled “Manipulation of Microparticles In Microfluidic Systems,” by Mehta et al. Complete integrated systems with fluid handling, signal detection, sample storage and sample accessing are available. For example, U.S. Pat. No. 5,942,443 provides pioneering technology for the integration of microfluidics and sample selection and manipulation.

A. General Fluid Flow Techniques in Microfluidic Devices

In general, enzymes, cells, modulators and other components can be flowed in a microscale system by electrokinetic (including either electroosmotic or electrophoretic) techniques, or using pressure-based flow mechanisms, or combinations thereof. In the present system, a combination of electrokinetic transport and pressure-based transport is typically used. For example, pressure is optionally used in a main channel to flow sample and buffer plugs through the main channel of an emulator device and electrokinetic transport is used, e.g., across side channels to create sample and plug plugs.

Electrokinetic material transport systems, e.g., electrokinetic controllers or electrokinetic fluid control elements, are used in microfluidic devices, e.g., planar, sipper, and emulator devices, to provide movement of samples, enzymes, substrates, modulators, and the like, through microfluidic channels, e.g., using an electrokinetic gradient set up across a channel or channel junction. “Electrokinetic material transport systems,” as used herein, include systems that transport and direct materials within a microchannel and/or chamber containing structure, through the application of electrical fields to the materials, thereby causing material movement through and among the channel and/or chambers, i.e., cations will move toward a negative electrode, while anions will move toward a positive electrode. For example, movement of fluids toward or away from a cathode or anode can cause movement of proteins, nucleic acids, enzymes, cells, modulators, etc. suspended within the fluid. Similarly, the components, e.g., proteins, antibodies, carbohydrates, etc. can be charged, in which case they will move toward an oppositely charged electrode (indeed, in this case, it is possible to achieve fluid flow in one direction while achieving particle flow in the opposite direction). In this embodiment, the fluid can be immobile or flowing and can comprise a matrix as in electrophoresis. For example, proteins are separated based on mass/charge ratio in a channel comprising a separation matrix, such as polyacrylamide.

Typically, the electrokinetic material transport and direction systems of the invention rely upon the electrophoretic mobility of charged species within the electric field applied to the structure. Such systems are more particularly referred to as electrophoretic material transport systems. For example, in the present system, separation of a mixture of components into its individual components optionally occurs by electrophoretic separation. For electrophoretic applications, the walls of interior channels of the electrokinetic transport system are optionally charged or uncharged. Typical electrokinetic transport systems are made of glass, charged polymers, and uncharged polymers. The interior channels are optionally coated with a material that alters the surface charge of the channel. In the present invention, channels in an emulator device are optionally fabricated to emulate a sipper device, e.g., by using the same charged or uncharged polymers and/or channel coatings.

A variety of electrokinetic controllers and systems which are optionally used in the present invention are described, e.g., in Ramsey WO 96/04547, Parce et al. WO 98/46438 and Dubrow et al., WO 98/49548, as well as a variety of other references noted herein.

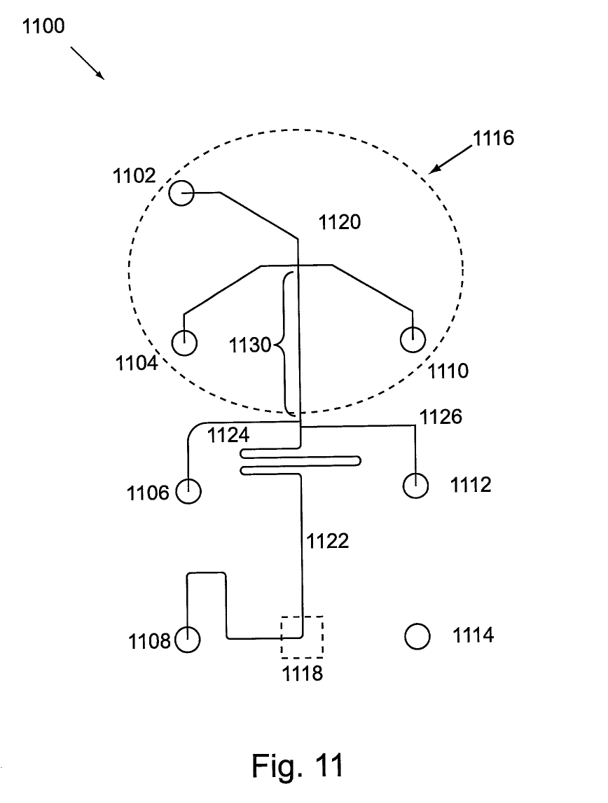

Use of electrokinetic transport to control material movement in interconnected channel structures was described, e.g., in WO 96/04547 and U.S. Pat. No. 5,858,195 by Ramsey. An exemplary controller is described in U.S. Pat. No. 5,800,690. Modulating voltages are concomitantly applied to the various reservoirs to affect a desired fluid flow characteristic, e.g., continuous or discontinuous (e.g., a regularly pulsed field causing the sample to oscillate direction of travel) flow of labeled components in one or more channels toward a waste reservoir. Particularly, modulation of the voltages applied at the various reservoirs, such as reservoirs 1102, 1104, 1110, and the like in FIG. 11, can move and direct fluid flow through the interconnected channel structure of the device, e.g., through capillary emulator 1116 into main channel region 1130.

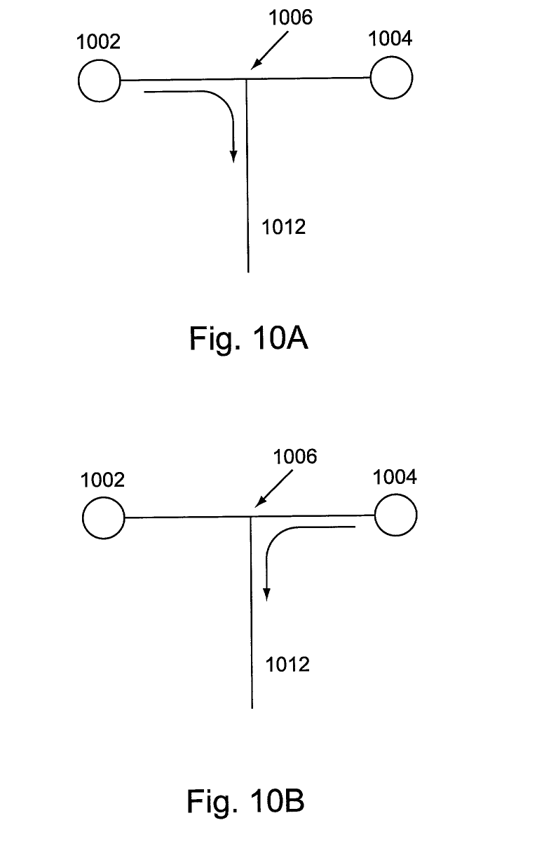

Other methods of transport are also available for situations in which electrokinetic methods are not desirable. For example, sample introduction and reaction are best carried out in a pressure-based system and high throughput systems typically use pressure induced sample introduction. In addition, cells are desirably flowed using pressure based flow mechanisms. In some embodiments of the present invention, pressure based fluid control is used to create sample and buffer plugs, thereby emulating sipper device fluid flow. See, e.g., FIG. 10.

Pressure based flow is also desirable in systems in which electrokinetic transport is also used. For example, pressure based flow is optionally used for introducing and reacting reagents in a system in which the products are electrophoretically separated. In the present system, a combination of pressure based flow and electrokinetic based flow is typically used to create sample and buffer plugs in an emulator device, e.g., a planar device, to emulate the flow of samples in a sipper device.

Pressure is optionally applied to microscale elements to achieve fluid movement using any of a variety of techniques. Fluid flow (and flow of materials suspended or solubilized within the fluid, including cells or other particles) is optionally regulated by pressure based mechanisms or pressure based fluid control elements, e.g., as part of fluid direction or control system, such as those based upon fluid displacement, e.g., using a piston, pressure diaphragm, vacuum pump, probe, or the like to displace liquid and raise or lower the pressure at a site in the microfluidic system. The pressure is optionally pneumatic, e.g., a pressurized gas, or uses hydraulic forces, e.g., pressurized liquid, or alternatively, uses a positive displacement mechanism, i.e., a plunger fitted into a material reservoir, for forcing material through a channel or other conduit, or is a combination of such forces.

In some embodiments, a vacuum source is applied to a reservoir or well at one end of a channel to draw the suspension through the channel. For example, a vacuum source is optionally placed at a reservoir in the present devices for drawing fluid into a channel, e.g., a vacuum source at reservoir 802 in FIG. 8 applies a pressure to main channel 812, thus drawing fluid, e.g., from reservoir 808, 810, or the like, into main channel 812.

Pressure or vacuum sources are optionally supplied external to the device or system, e.g., external vacuum or pressure pumps sealably fitted to the inlet or outlet of the channel, or they are internal to the device, e.g., microfabricated pumps integrated into the device and operably linked to the channel. Examples of microfabricated pumps have been widely described in the art. See, e.g., published International Application No. WO 97/02357.

Another alternative to electrokinetic transport is an electroosmotic pump which uses electroosmotic forces to generate pressure based flow. See, e.g., published International Application No. WO 99/16162 by Parce, entitled “Micropump.” An electroosmotic pump typically comprises two channels. The pump utilizes electroosmotic pumping of fluid in one channel or region to generate pressure based fluid flow in a connected channel, where the connected channel has substantially no electroosmotic flow generated. For example, an electrokinetic controller applies a voltage gradient to one channel to produce electroosmotically induced pressure within that channel. That pressure is transmitted to a second channel whereupon pressure based flow is achieved. In the present invention, an electroosmotic pump is optionally used to produce pressure-based flow, e.g., in the main channel. The channel surfaces of the pumping channel typically have charged functional groups associated therewith to produce sufficient electroosmotic flow to generate pressure in the channels in which no electroosmotic flow takes place. See WO 99/16162 for appropriate types of functional groups.

Hydrostatic, wicking, and capillary forces are also optionally used to provide pressure for fluid flow of materials such as enzymes, substrates, modulators, or protein mixtures. See, e.g., “METHOD AND APPARATUS FOR CONTINUOUS LIQUID FLOW IN MICROSCALE CHANNELS USING PRESSURE INJECTION, WICKING AND ELECTROKINETIC INJECTION,” by Alajoki et al., U.S. Ser. No. 09/245,627, filed Feb. 5, 1999. In these methods, an absorbent material or branched capillary structure is placed in fluidic contact with a region where pressure is applied, thereby causing fluid to move towards the absorbent material or branched capillary structure. The capillary forces are optionally used in conjunction with the electrokinetic or pressure-based flow in the present invention. The capillary action pulls material through a channel. For example a wick is optionally added to a main channel to aid fluid flow by drawing liquid, e.g., sample and/or buffer plugs, through the channel.

Mechanisms for reducing adsorption of materials during fluid-based flow are described in “PREVENTION OF SURFACE ADSORPTION IN MICROCHANNELS BY APPLICATION OF ELECTRIC CURRENT DURING PRESSURE-INDUCED FLOW” U.S. Ser. No. 09/310,027, filed May 11, 1999 by Parce et al. In brief, adsorption of cells, components, proteins, enzymes, and other materials to channel walls or other microscale components during pressure-based flow can be reduced by applying an electric field such as an alternating current to the material during flow.

Mechanisms for focusing cells, enzymes, and other components into the center of microscale flow paths, which are useful in increasing assay throughput by regularizing flow velocity, e.g., in pressure based flow, are described in “FOCUSING OF MICROPARTICLES IN MICROFLUIDIC SYSTEMS” by H. Garrett Wada et al., U.S. Ser. No. 09/569,747, filed May 11, 1999. In brief, sample materials are focused into the center of a channel by forcing fluid flow from opposing side channels into a main channel comprising the sample materials, or by other fluid manipulations.

In an alternate embodiment, microfluidic systems are incorporated into centrifuge rotor devices, which are spun in a centrifuge. Fluids and particles travel through the device due to gravitational and centripetal/centrifugal pressure forces. For example, samples are optionally transported through a main channel of a planar device using centrifugal force. In emulator devices, sample plugs are optionally transported in this manner.

The above fluid transport techniques for microfluidic devices are optionally integrated into one device. For example, in the non-sipper devices of the invention, e.g., planar devices, pressure based control elements are optionally used to mix and react various components and electrokinetic control elements are optionally used to separate products that result from such a reaction. In emulator devices, which are described in more detail below, electrokinetic flow is optionally used to load sample and buffer plugs and pressure-based flow is optionally used to transport those plugs through the channels of the device.

In addition to transport through the microfluidic system, the invention also provides for introduction of sample or reagents, e.g., enzymes, proteins, substrates, modulators, and the like, into the microfluidic system.

Reservoirs or wells are provided in the present invention, e.g., in non-sipper and sipper devices, as sources of samples, reagents, enzymes, substrates, nucleic acids, buffers, and the like. Such wells include, e.g., reservoirs 802, 804, 806, 808, and 810 in FIG. 8, which illustrates a non-sipper device. The wells are typically disposed on or within the body structure of the device. For example, a sample is optionally introduced into the device through reservoir 808, e.g., using a pipette. These reservoirs comprise internal sources of samples and reagents. In other embodiments, the reservoirs are used to introduce samples via a pipette from an external source.

The above devices, systems, features, and components are used as described below, e.g., to separate a mixture of components, to perform enzyme assays, to separate substrates and products, to separate and/or sequence nucleic acids, to perform PCR, to screen a drug library, to perform fluorescence polarization assays, mobility shift assays, and the like. The planar devices are typically used in a benchtop method to perform an assay, e.g., on one or a few samples. Microfluidic sipper devices are typically used when high throughput methods are desired, e.g., for drug screening. The sipper devices are described in more detail below, followed by a description of an emulator device of the invention. The emulator devices are used to emulate the fluid flow profile of a sipper device on a planar device, e.g., so assays can be designed and optimized on a bench-top planar system before performing them on a high throughput scale.

II. Microfluidic Sipper Devices

Microfluidic sipper devices are typically used in high throughput applications, e.g., drug screening or any application with a large number of samples to be tested. A “microfluidic device comprising an external capillary” is used herein to refer to a microfluidic device, e.g., as described in the references cited above, that has a fluidly coupled external source of reagents. For example, a microfluidic sipper device is device with an external capillary, e.g., a small tube inserted into a channel or reservoir of the device. “Microfluidic sipper device,” “sipper device,” and “sipper substrate” are used herein to refer to a device comprising an external capillary. The capillary is typically an external channel or capillary, pipettor channel or the like that is coupled to a channel disposed within the body structure of the device. An “external capillary” in the present invention is one that extends from the body of a microfluidic device, as opposed to the internal channels that are disposed within the body structure.

A microfluidic sipper device typically comprises an external capillary coupled to one or more channel disposed within the body structure of the device. In addition to an external capillary, sipper devices also optionally comprise one or more reservoirs and internal channels, e.g., the channels disposed within the body structure. The body structure, internal channels, e.g., channels disposed within the body of the device, and reservoirs, are typically the same or similar to those described above for planar devices. The channels may, however, have different sizes, e.g., lengths, widths, and/or depths, different coatings, or the like. The channels are used to flow fluidic materials through the device and the reservoirs are used, e.g., to store reagents, e.g., for use in assays, or for waste disposal.

In the present invention “sipper main channel” and “sipper side channel” are used to distinguish between similar channels in a sipper device and a non-sipper device. A “sipper main channel” is typically a microfluidic main channel within a device comprising an external capillary, e.g., a sipper capillary. A “sipper side channel” is typically a microfluidic side channel within a device comprising a sipper capillary. The channels in the sipper devices and in the non-sipper, e.g., planar, devices, are typically similar but may comprise different lengths, widths, depths, flow characteristics, resistances, coatings, and the like.

Sources of samples, mixtures of components, and reagents, e.g., enzymes, substrates, and the like, are fluidly coupled to the microchannels in a sipper device in any of a variety of ways. In particular, those systems comprising sources of materials set forth in Knapp et al. “Closed Loop Biochemical Analyzers” (WO 98/45481; PCT/US98/06723) and Parce et al. “High Throughput Screening Assay Systems in Microscale Fluidic Devices” WO 98/00231 and, e.g., in 09/510,626 filed Feb. 22, 2000, entitled “Manipulation of Microparticles In Microfluidic Systems,” by Mehta et al. are applicable.

In these systems, a “pipettor channel” or “external capillary” (a channel in which components can be moved from a source to a microscale element such as a second channel or reservoir) is temporarily or permanently coupled to a source of material. The source can be internal or external to a microfluidic device comprising the pipettor channel. An external source is one that is not included within the body of the device. Example sources include microwell plates, membranes or other solid substrates, e.g., comprising lyophilized components, wells or reservoirs in the body of the microscale device itself, and others. These types of sample and reagent sources are typically used with what is referred to herein as a sipper device. The sipper devices typically comprise an external capillary as well as internal reservoirs as described above. For example, a microfluidic sipper device optionally comprises an external capillary, e.g., for introducing samples from a microwell plate or membrane, and one or more internal reservoirs for introducing additional reagents, e.g., stored in lyophilized form, in the reservoirs of a sipper device.

For example, the source of a sample, component, or buffer can be a microwell plate or other container or solid phase structure external to the body structure, having, e.g., at least one well with the selected reagent, test material, buffer, sample, or component, e.g., in fluidic form, or lyophilized, or otherwise in dried form.

A loading channel region is optionally fluidly coupled to a pipettor channel with a port external to the body structure. The loading channel can be coupled to an electropipettor channel with a port external to the body structure, a pressure based pipettor channel with a port external to the body structure, a pipettor channel with a port internal to the body structure, an internal channel within the body structure fluidly coupled to a well on the surface of the body structure, an internal channel within the body structure fluidly coupled to a well within the body structure, or the like.

Integrated microfluidic systems of the invention optionally include a very wide variety of storage elements for storing samples and reagents to be assessed. These include well plates, matrices, membranes, and the like. The reagents are stored in liquids (e.g., in a well on a microtiter plate), or in lyophilized form (e.g., dried on a membrane or in a porous matrix), and can be transported to an array component, region, or channel of the microfluidic device using conventional robotics, or using an electropipettor or pressure pipettor channel fluidly coupled to a region or channel of the microfluidic system. These systems are optionally used with the planar devices of the invention or with the sipper devices of the invention.

A. Fluid Flow in a Microfluidic Device Comprising an External Capillary

In general, fluid is flowed through a sipper device in the same manner as described above for planar devices, e.g., electrokinetically, driven by pressure, or a combination thereof. However, samples are introduced into a sipper device in a different manner than in a planar device. A sample is typically pipetted into a reservoir in a planar device and is typically sipped into the channels of the device via the external capillary or sipper in a sipper device. In the planar device, the sample is typically flowed continuously from the reservoir into the channel of interest. In the sipper devices, a portion of sample or a sample plug is sipped, e.g., from an external source such as a microwell plate or LibraryCard™ to create discrete sample plugs. Multiple samples are sipped, e.g., interspersed with a buffer material or other spacer material, to create a series of sample plugs separated by buffer plugs or spacer plugs.

The sipping of discrete sample plugs yields a different fluid flow profile than that typically achieved in a planar device. The different fluid flow profiles makes scaling up from planar device to a high throughput sipper device, e.g., for automation and/or streamlining, difficult. The emulator devices and methods described below alleviate this problem by emulating the fluid flow profile of the sipper device in a planar device, e.g., a benchtop non-sipper device.

A “fluid flow profile” in the present invention, refers to the manner in which fluidic materials are flowed through a microfluidic device. The profile typically refers to such things as flow rate and format, e.g., how a fluid is transported through a channel system including, but not limited to, the order and type of materials. In some embodiments, the fluid flow profile includes various flow characteristics, such as flow rate, channel resistance, e.g., hydrodynamic resistance, order of materials flowed through a channel, distance materials are transported, and the like. “Flow characteristic” is typically used to refer to different aspects of the fluid flow a material in a channel, e.g., how much time the fluid takes to travel a certain distance, how fast a fluid flows, how much resistance, e.g., hydrodynamic or electrical, a fluid encounters in a channel, how much dispersion and/or diffusion a material undergoes as it flows through a certain channel, and the like.

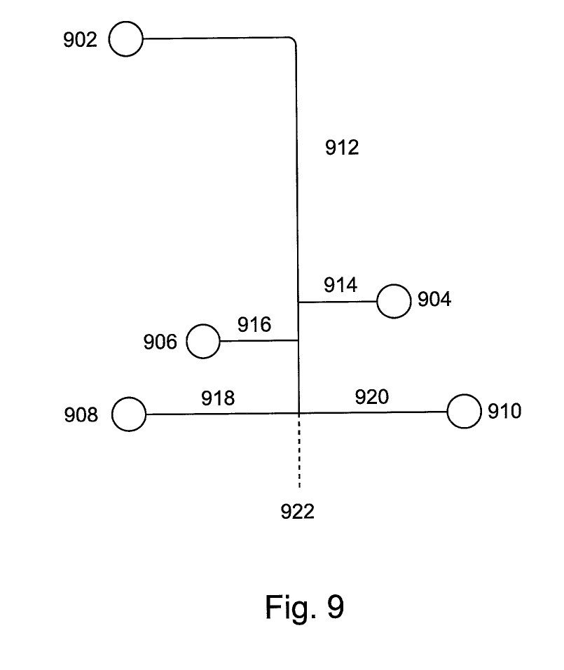

For example, one embodiment of a sipper microfluidic device is illustrated in FIG. 9. As shown, the system comprises reservoirs 902, 904, 906, and 910 disposed within body structure 900, which are optionally used to introduce samples and/or reagents into the system. For example, reservoir 908 is optionally used to introduce a reagent into main channel 912, e.g., through side channel 918. A reagent is optionally stored, e.g., a pre-disposed or dried reagent, in a reservoir, e.g., reservoir 908 for introduction into the device.

In addition to the internal reservoirs, sipper device 900 comprises external capillary 922 extending from body structure 900 and fluidly coupled to main channel 912. External capillary 922 is used to draw or sip channels from an external source, e.g., a microwell plate or solid substrate, such as a LibraryCard™. For example, a microwell plate comprising a plurality of test compounds and a buffer well is placed beneath external capillary 922, e.g., using a conveyor system running beneath device 900. External capillary 922 sips a first test compound or sample from a first position, e.g., in an array or microwell plate, and then sips a buffer sample into the capillary, e.g., under pressure, e.g., applied by a vacuum at reservoir 902. The capillary is then used to sip a second test compound and then a buffer and so on, thereby creating a series of sample plugs separated by buffer plugs. The sample and buffer plugs are transported through the device, e.g., under pressure or using electrokinetic forces, as described above.

Upon introduction into the device, the sample plugs and buffer plugs are typically flowed, e.g., from external capillary 922 through main channel 912. In some embodiments, the sample is optionally diluted, e.g., with a buffer aliquot added from reservoir 910 through side channel 920. Typically, samples are mixed with reagents in main channel 912. For example, samples in main channel 912 are optionally mixed with reagents from reservoirs 906 and 904, which are added, e.g., in a continuous stream, through side channels 916 and 914. For example a DNA sample is optionally mixed with PCR reagents in main channel 912. Fluid flow in main channel 912 is typically controlled by a pressure source, e.g., a pressure source fluidly coupled to either reservoir 908 or 910, or waste reservoir 902, or electrokinetic forces applied at various reservoirs, e.g., using electrodes at reservoirs 908, 910, 906, 904, and 902. For example, a vacuum source is optionally coupled to waste reservoir 902. In other embodiments, an additional channel, e.g., a separation channel such as a polyacrylamide filled channel, is included in the planar device to separate various components, e.g., PCR products. A detector is optionally positioned proximal to waste reservoir 902 or proximal to the downstream end of main channel 912 to detect components as they flow through the channel, e.g., at a detection window. When the assay and detection are complete, the sample plugs, e.g., reacted sample plugs, and buffer plugs are optionally directed to waste reservoir 902 for disposal or retrieval. Any of the reservoirs, e.g., 902, 904, 906, 908, and 910 are optionally used as waste wells.

In the present invention, emulator devices typically emulate at least one flow characteristic or at least one aspect of the fluid flow profile for a fluid on a sipper device, e.g., discrete sample plugs, equivalent reaction times, and the like. In one embodiment, emulator devices emulate the fluid flow profile of a sipper device, such as that in FIG. 9, by creating alternating buffer plugs and sample plugs in the main channel of the device. Various methods of creating such flow profiles without the use of an external capillary are described below.

III. Emulator Devices

Emulator devices of the present invention typically comprise microfluidic devices that do not comprise a functional external capillary, in other words, planar devices. However, emulator devices differ from the planar devices described above because they are fabricated to emulate or copy the fluid flow profile of a sipper device. The devices are used to emulate the fluid flow profile of a device that comprises an external capillary, e.g., a sipper device. Fluid flow in a typical planar device does not typically produce to a fluid flow profile comprising alternating sample plugs and buffer plugs or spacer plugs. Alternating sample plugs and buffer plugs are a typical aspect of sipper device fluid flow profiles, in which an external capillary creates sample and buffer plugs by alternately drawing fluid, e.g., from a sample source and a buffer source. Emulator devices of the present invention are planar devices that are used or configured in such a way as to create a similar fluid flow profile, e.g., alternating sample and buffer plugs. The emulator devices therefore function as a planar equivalent of a sipper device and can be used to optimize an assay in a benchtop format, e.g., for later automation or streamlining in a sipper device high throughput format.

The emulator devices are used, e.g., to design, optimize, and/or debug assays on planar devices, e.g., planar devices used at a benchtop microscope station. Emulator devices typically function in one of two ways. In one embodiment, planar devices are fabricated that have substantially identical hydrodynamic and electrical resistances as their sipper device equivalents, e.g., the devices they emulate. In these devices, the capillary channel in a sipper device is represented as a channel, e.g., an emulator channel, on the planar emulator device, e.g., an isotropically etched channel, having a substantially equivalent hydrodynamic resistance as an external capillary on a sipper device. The emulator channel is used, e.g., to introduce samples into a main channel for analysis. The planar equivalent of the sipper device, e.g., the emulator device, is optionally used in steady state mode, e.g., the flow rates of various components, e.g., from a reservoir to a main channel via side channel, does not vary with time. In a second embodiment, compound sipping, as commonly used in sipper devices, is simulated in an emulator device using an electrokinetic injector. The injector typically operates in a dual-mode manner, e.g., using both pressure based flow and electrokinetic flow. The electrokinetic injector is used to optimize flow characteristics, such as sample and buffer sip timing, to emulate the sample and buffer plugs of sipper devices.

In a dual mode injector emulator, one or more sample plugs and/or buffer plugs are generated by loading a sample from a source, e.g., an internal reservoir, into a channel of the emulator device, e.g., a main channel of a non-sipper device, followed by loading a buffer into the channel of the emulator device, e.g., from an internal source or reservoir.

In one embodiment, loading the sample into a channel of the device from an internal reservoir comprises applying an electrokinetic gradient between a source of the sample, e.g., an internal reservoir, and another internal reservoir, e.g., a waste reservoir. Loading the buffer optionally comprises applying an electrokinetic gradient between a source of the buffer and another reservoir, e.g., the waste reservoir. For example, to use the planar device pictured in FIG. 8 as an emulator device, sample is optionally loaded from reservoir 808 by applying an electrokinetic gradient between reservoir 808 and waste reservoir 802. Loading the buffer comprises applying a gradient between reservoir 810, e.g., a buffer source, and waste reservoir 802. As the gradients are applied, the sample or buffer move into main channel 812 and are thereby loaded into the device. When the gradients are alternately and repeatedly applied, the loading results in a series of sample plugs separated by buffer plugs. The electrokinetic gradients used to load the buffer and the sample are optionally different or substantially equal, e.g., in strength. In addition, the gradients may be adjusted to optimize the size and timing of the sample plugs and buffer plugs, e.g., to emulate sipping of the plugs in a microfluidic sipper device.

In other embodiments, sample and buffer are alternately loaded into a channel by alternately applying pressure to a sample reservoir and to a buffer reservoir. The buffer and sample are typically alternately loaded into the channel and flowed through the device, e.g., to be assayed or reacted with other components.

Alternating sample and buffer plugs are typically flowed through a channel in an emulator device by applying pressure to the sample and/or the buffer in the channel. After loading the sample plugs and buffer plugs into a channel, e.g., by applying pressure or electrokinetic gradients, the plugs are typically flowed through the channel under pressure. For example, a pressure is optionally applied continuously on a main channel while sample plugs and buffer plugs are alternately loaded into the main channel, so that as each plug, e.g., sample or buffer, is loaded into the channel, it is immediately subjected to a pressure and moves through the channel. The pressure applied to the channel to transport sample and buffer are optionally the same or different pressures, e.g., depending on the desired size and timing for each plug.

For example, in FIG. 8, a pressure is optionally continuously applied to main channel 812, e.g., from a vacuum at waste reservoir 802. The pressure is applied to transport sample plugs and buffer plugs, created as described above, through main channel 812. For example, an electrokinetic gradient is optionally applied alternately between reservoir 802 and sample reservoir 808 and between reservoir 802 and buffer reservoir 810 to create alternating sample and buffer plugs. A pressure is simultaneously applied to the sample and buffer plugs in channel 812, e.g., via a pressure based fluid control element coupled to reservoir 802. By emulating the fluid flow profile of a sipper device, the planar device in FIG. 8 becomes an emulator device.

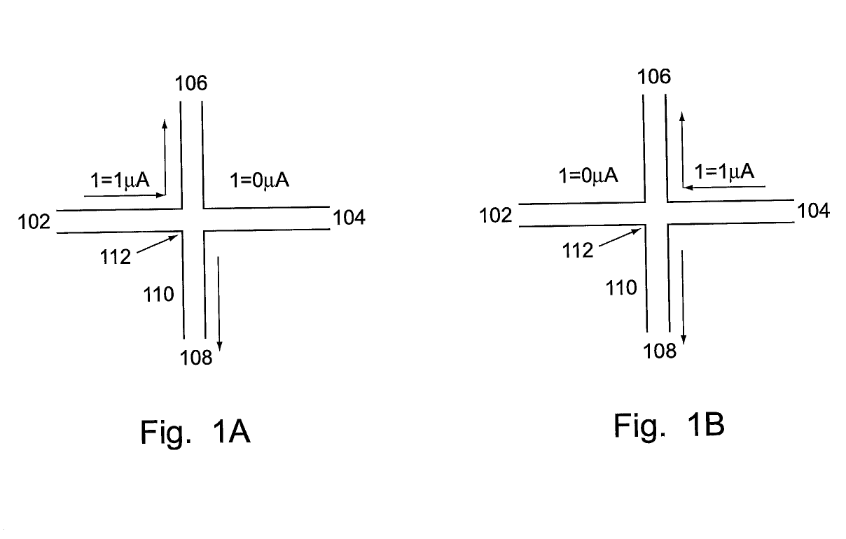

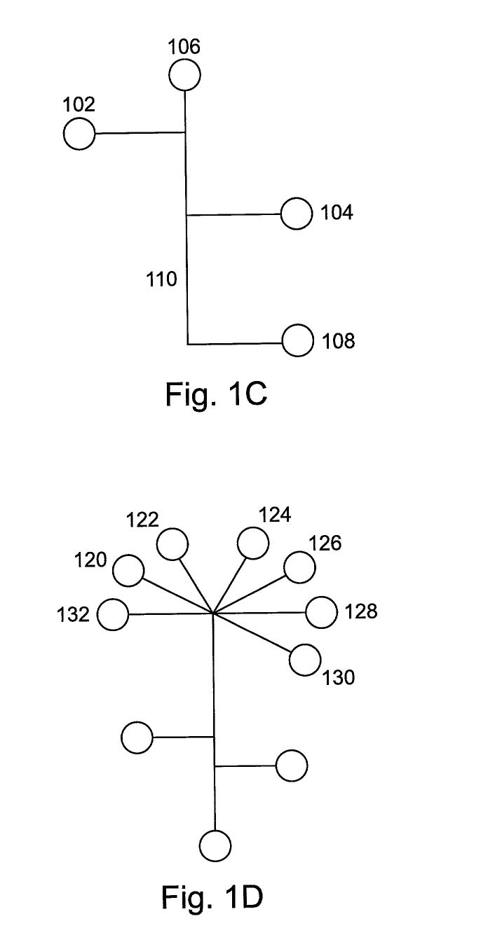

For example, FIG. 1 illustrates a dual mode injector, which is used herein to create buffer and sample plugs in a planar device. The injector comprises crossjunction 112 with sample and buffer being supplied from opposite sides of the cross, e.g., sample well 102 and buffer well 104. The other two arms of the cross are connected to vacuum and waste wells, e.g., vacuum well 108 and waste well 106. These wells are typically internal reservoirs, e.g., internal to the body structure of a non-sipper device. Sample pulses, e.g., of arbitrary duration are generated by switching between two electrokinetic flow states while maintaining a steady vacuum at vacuum well 108. In state A, illustrated by Panel A, electrokinetic pumping is used to transport sample to waste by placing a potential difference between sample well 102 and waste well 106, e.g., a low current, e.g., about 1 mA. Substantially zero current is applied between buffer well 104 and waste well 106 during this time. The applied voltage drives sample material from sample well 102 through cross junction 112, while no buffer is drawn into the junction area, thus creating an undiluted sample plug for delivery to main channel 110. Concurrently with the voltage being applied, the vacuum pressure at well 108 draws the sample at the cross-junction into main channel 110. To terminate the delivery of the sample, e.g., with a buffer spacer, the electrokinetic flow is switched to state B. Panel B illustrates state B in which the electrokinetic flow is switched to provide a voltage application between buffer well 104 and waste well 106, which drives buffer material into cross-junction 112. No sample is flowed into the junction area during this state, because the electrokinetic flow between sample well 102 and waste well 106 has been stopped. However, pressure is concurrently applied to main channel 110 to transport the buffer material at cross-junction 112 into and through main channel 110. Typically, the electrokinetic forces used to load the sample and buffer plugs are substantially larger than the forces used to drive pressure flow. Therefore, the fluid composition at junction 112 is controlled by electrokinetic flow, thereby creating undiluted sample and buffer plugs.

FIG. 1, Panels C and D, illustrates alternate channel configurations for dual mode injectors. For example, Panel C illustrates a dual-mode injector that does not form a typical cross intersection. In a device of Figure C, sample is optionally contained in reservoir 102 and buffer in reservoir 104. Reservoir 106 is typically used for waste and a vacuum is applied at reservoir 108. Electrokinetic and pressure forces are typically used to load sample plugs into main channel 110, e.g., by alternately applying voltage between reservoir 102 and reservoir 106 and between reservoir 104 and 106, while continuously applying a pressure gradient along channel 110. In Figure D, the channel configuration provides multiple sample sources or reservoirs. For example reservoirs 120, 122, 124, 126, 128, and 130 each optionally comprises a different sample and reservoir 132 comprises buffer. The samples are loaded as described above, e.g., ten sample plugs of each sample are optionally loaded into a main channel using alternate applications of current between sample reservoirs and waste reservoirs and between buffer reservoirs and waste reservoirs. These channel configurations are shown for purposes of illustration. However, those of skill in the art will recognize other configurations that are optionally used to provide dual mode injection of one or multiple samples in a planar device, e.g., to emulate sipper device fluid flow.

In another embodiment, a capillary emulator channel is used to emulate a sipper fluid flow profile in a non-sipper device. A capillary emulator channel is typically a channel in a planar device that simulates an external capillary channel, e.g., a sipper channel. The capillary emulator channel typically simulates the external capillary by providing substantially the same hydrodynamic resistance as an external capillary, e.g., in a sipper device. In addition, the capillary emulator channel optionally has substantially the same length, width, and/or depth as an external capillary. Typically, a capillary emulator channel flows substantially the same amount of fluid in substantially the same amount of time as an external capillary.

To emulate a sipper device fluid flow profile using a capillary emulator channel, a sample is typically flowed from an internal source into a non-sipper main channel via the capillary emulator channel. The non sipper main channel is a main channel in a non-sipper device that simulates fluid flow of a main channel of a sipper device as described above, e.g., by providing substantially the same amount of fluid flow in substantially the same amount of time, by providing substantially the same hydrodynamic resistance, or the like.

Reagents, buffers, and the like or optionally added to the sample in the non-sipper main channel, e.g., via a non-sipper side channel, from a reservoir, e.g., an internal reservoir. For example, a drug sample or other test sample is optionally flowed through a capillary emulator channel into a non-sipper main channel. Various reagents, e.g., enzymes against a drug is to be tested, are then added from an internal reservoir to the main channel via a non-sipper side channel, to react with the test compound or sample. The non-sipper side channel simulates a side channel in a sipper device as described above. The sample is typically allowed to incubate with the reagent or enzyme of interest, e.g., for a specified amount of time. The non-sipper channels, e.g., the main channel and capillary emulator channel, are configured to provide the same flow rates, resistance, and incubation time as equivalent channels in a sipper device, e.g., the sipper main channel and a sipper capillary, thereby emulating the fluid flow profile of the sipper device.

A. Emulator Devices and Methods of Fabrication

Emulator devices are provided in one embodiment of the present invention. These devices are used as assay development devices, e.g., to design, optimize, and debug assays, e.g., prior to scaling up an assay to a high throughput system. The assay devices of the invention are typically planar or non-sipper microfluidic devices, e.g., microfluidic devices that do not comprise an external capillary.

The devices typically comprise a microfluidic substrate, e.g., a planar substrate, with a plurality of microfluidic channels disposed therein. The channels typically comprise a main channel and a capillary emulator fluidly coupled to the main channel. In addition, the devices comprise at least a first fluid control element, e.g., a pressure source, an electrokinetic controller, or both, fluidly coupled to the main channel.

The main channel in the emulator devices of the invention typically simulates the main channel of a sipper device. For example, the emulator main channel typically comprises a hydrodynamic resistance, length, width, depth, or flow characteristic that is substantially equal to a sipper device main channel. For example, an emulator main channel typically transports substantially the same amount of fluid in substantially the same amount of time as a non-sipper main channel. The main channel of the emulator device is typically used to perform an assay on one or more sample or test compound, e.g., a protease reaction.

The capillary emulator typically comprises a microscale channel, which comprises substantially the same hydrodynamic resistance, length, width, depth, and/or flow characteristics, as a sipper capillary in a microfluidic sipper device. The capillary emulator is typically used to introduce samples and or buffer materials into the main channel of the emulator device, e.g., to mimic the fluid flow profile of a sipper device, e.g., by creating a sample plug the same size, e.g., same length of a sample plug sipped through a capillary.

In another embodiment, the capillary emulator comprises a waste reservoir, fluidly coupled to the main channel of the device, and a sample reservoir, fluidly coupled to the waste reservoir and the main channel. In addition, the capillary emulator comprises a buffer well fluidly coupled to the waste reservoir and the main channel. The fluid control elements transport fluid in the emulator device, e.g., through the capillary emulator to emulate the sipping of compounds into a sipper device, e.g., through an external capillary from a microwell plate or LibraryCard™.

For example, the fluid control element optionally comprises a pressure source and an electrokinetic controller. The pressure source applies a pressure differential between the waste reservoir and the pressure source. The electrokinetic controller alternately applies an electrokinetic gradient between the sample well and the waste reservoir and between the buffer well and the waste reservoir. Typically, the pressure source and the electrokinetic controller function substantially simultaneously to simulate the sipping of sample and buffer plugs as described above.

In other embodiments, the fluid control element comprises one or more pressure source and the capillary emulator comprises a buffer well and a sample well fluidly coupled to the main channel. The fluid control element applies a first pressure to a sample in the sample well and a second pressure to a buffer material in the buffer well. The application of pressure to the wells transports a portion of a sample into the main channel and a portion of buffer into the main channel. For example, the first pressure and the second pressure are typically applied alternately to create sample plugs and buffer plugs. Typically, when a pressure is applied to a sample well to flow that sample into the main channel, the pressure at the buffer well is maintained at a specified pressure to prevent buffer from flowing into the main channel with the sample. To flow sample and buffer alternately without mixing the two materials, the reservoir which is not being loaded is maintained at a node pressure, e.g., the pressure that exists at the junction between the well and the main channel. See, e.g., FIG. 10, which illustrates the creation of sample and buffer plugs in an emulator device. FIG. 10, Panel A, shows a sample being loaded in an emulator device. A sample is loaded from sample reservoir 1002 by applying a pressure P1 at sample reservoir 1002. The pressure at junction 1006 is P0 and the pressure at buffer well 1004 is maintained at P0 also. The pressure P1 forces fluid from sample well 1002 into main channel 1012, where it is transported through the channel, e.g., for reaction. Panel B illustrates the loading of a buffer plug into the main channel. A pressure, P1, is applied at buffer well 1004 and a second pressure, P0 is applied at sample well 1002 to maintain the sample well pressure at a pressure substantially equal to the pressure at junction 1006, e.g., to prevent the sample from contaminating the buffer plug. Typically, the fluid control element applies a third pressure to the sample and/or buffer in the main channel concurrent to the application of the first and second pressures, e.g., alternately applied first and second pressures. The pressure in the main channel transports the sample and buffer plugs through the main channel of the emulator device.

Emulator devices, as described above, are typically fabricated by providing a non-sipper microfluidic substrate, e.g., a planar microfluidic substrate without an external capillary and fabricating two or more channels within the microfluidic substrate. For example, the two or more channels optionally comprise a capillary emulator, a main channel, a side channel, and/or a reservoir. The two channels are typically fabricated to emulate an external capillary and a main channel of a microfluidic sipper device. For example, the channels are fabricated to emulate an external capillary and main channel of a microfluidic sipper device by providing substantially the same hydrodynamic resistance, substantially the same width, substantially the same depth, substantially the same length, or substantially the same flow characteristics as the external capillary or the main channel of the microfluidic sipper device. Having substantially the same flow characteristics as a channel or capillary in a microfluidic sipper device typically comprises providing substantially the same amount of fluid flow in substantially the same amount of time.

In other embodiments, the emulator devices of the invention may include an external capillary, e.g., a non-functional capillary or a capillary that does not function as a sample introduction element. In addition, the sipper devices of the invention are optionally used in the manner described above for emulator devices, e.g., without a functioning sipper capillary. For example, a sipper device is optionally loaded and samples loaded and flowed through the device as described above, instead of using the sipper capillary to introduce the samples.

After fabrication of channels that emulate sipper channels, the emulator devices are used as described above to emulate a fluid flow profile of a sipper device in a planar device. The devices are then used to develop assays on planar systems that can be efficiently used in a sipper device as well, e.g., for high throughput applications. Emulator devices are optionally incorporated into integrated systems as described below. In addition, example emulator systems are also illustrated below.

B. Integrated Systems

Although the devices and systems specifically illustrated herein are generally described in terms of the performance of a few or one particular operation, it will be readily appreciated from this disclosure that the flexibility of these systems permits easy integration of additional operations into these devices. For example, the emulator devices and systems described optionally include structures, reagents and systems for performing virtually any number of operations both upstream and downstream from the operations specifically described herein. Such upstream operations include sample handling and preparation operations, e.g., cell separation, extraction, purification, amplification, cellular activation, labeling reactions, dilution, aliquotting, and the like. Similarly, downstream operations may include similar operations, including, e.g., labeling of components, assays and detection operations, electrokinetic or pressure-based injection of components into contact with particle sets, or materials released from particle sets, or the like.

C. Instrumentation

In the present invention, materials such as cells, proteins, enzymes, or antibodies are optionally monitored so that a component of interest can be detected or identified or an activity can be determined. For example, after an enzyme assay, the amount of substrate and product is optionally quantitated based on the area of the detected signals. Depending on the detected signal measurements, decisions are optionally made regarding subsequent fluidic operations, e.g., whether to automate the assay in a high throughput format, e.g., on a sipper device, or whether to assay a particular component in detail to determine, e.g., kinetic information.

The systems described herein generally include microfluidic devices, e.g., emulator devices as described above, in conjunction with additional instrumentation for controlling fluid transport, flow rate and direction within the devices, detection instrumentation for detecting or sensing results of the operations performed by the system, processors, e.g., computers, for instructing the controlling instrumentation in accordance with preprogrammed instructions, receiving data from the detection instrumentation, and for analyzing, storing and interpreting the data, and providing the data and interpretations in a readily accessible reporting format. The planar emulator devices of the invention are optionally integrated with the above components in a single benchtop research station.

D. Fluid Direction System

A variety of controlling instrumentation is optionally utilized in conjunction with the microfluidic devices described above, for controlling the transport and direction of fluidic materials and/or materials within the devices of the present invention, e.g., by pressure-based or electrokinetic control as described above. In the present invention both pressure based and electrokinetic fluid control are often combined in an emulator device to create sample and buffer plugs to emulate the sipping of compounds in device with an external capillary.

Typically, the fluid controller systems provided are appropriately configured to receive or interface with a microfluidic device or system element as described herein. For example, the controller and/or detector, optionally includes a stage upon which the device of the invention is mounted to facilitate appropriate interfacing between the controller and/or detector and the device. Typically, the stage includes an appropriate mounting/alignment structural element, such as a nesting well, alignment pins and/or holes, asymmetric edge structures (to facilitate proper device alignment), and the like. Many such configurations are described in the references cited herein.

The controlling instrumentation discussed above is also used to provide for a dual-mode electrokinetic injector that switches fluid flow between two reservoirs in a planar device to emulate a sipper device. In addition, a constant pressure is typically applied concurrently to the electrokinetic injector to move the sample and buffer plugs created by the injector through the device. In other embodiments, pressure control at various wells, as described above, is controlled by the fluid direction system to emulate sipper fluid flow profiles in a planar device that has been fabricated to provide substantially similar flow rates, resistances, and the like, as the sipper device.

E. Detector

Once an assay has been performed, components, e.g., samples and products, are typically detected. For example, an enzyme reaction product and unreacted substrate that were separated in a separation channel of an emulator device are typically detected and quantitated. The detector(s) optionally monitors one or a plurality of signals, e.g., from a component of interest, e.g., in a main channel. For example, the detector optionally monitors an optical signal that corresponds to a labeled component, such as a labeled substrate or labeled product located in a detection region or detection channel, e.g., a detection region that is proximal to a waste reservoir.

Proteins, substrates, products, antibodies, or other components which emit a detectable signal, e.g., fluorescein labeled substrates or products, can be flowed past the detector, or, alternatively, the detector can move relative to the array to determine protein position (or, the detector can simultaneously monitor a number of spatial positions corresponding to channel regions, e.g., as in a CCD array).

The detector can include or be operably linked to a computer, e.g., which has software for converting detector signal information into assay result information, e.g., molecular weight based on retention time or elution time, identity of a protein, concentration of a substrate or product, or the like.

Examples of detection systems include optical sensors, temperature sensors, pressure sensors, pH sensors, conductivity sensors, and the like. Each of these types of sensors is readily incorporated into the emulator systems described herein. In these systems, such detectors are placed either within or adjacent to the microfluidic device or one or more channels, chambers or conduits of the device, such that the detector is within sensory communication with the device, channel, or chamber. The phrase “proximal,” to a particular element or region, as used herein, generally refers to the placement of the detector in a position such that the detector is capable of detecting the property of the microfluidic device, a portion of the microfluidic device, or the contents of a portion of the microfluidic device, for which that detector was intended. For example, a pH sensor placed in sensory communication with a microscale channel is capable of determining the pH of a fluid disposed in that channel. Similarly, a temperature sensor placed in sensory communication with the body of a microfluidic device is capable of determining the temperature of the device itself.

Particularly preferred detection systems include optical detection systems for detecting an optical property of a material within the channels and/or chambers of the microfluidic devices that are incorporated into the microfluidic systems described herein. For example, fluorescent or chemiluminescent detectors are typically preferred. Such optical detection systems are typically placed adjacent to a microscale channel of a microfluidic device, and are in sensory communication with the channel via an optical detection window that is disposed across the channel or chamber of the device. Optical detection systems include systems that are capable of measuring the light emitted from material within the channel, the transmissivity or absorbance of the material, as well as the material's spectral characteristics. In preferred aspects, the detector measures an amount of light emitted from the material, such as a fluorescent or chemiluminescent material. As such, the detection system will typically include collection optics for gathering a light based signal transmitted through the detection window, and transmitting that signal to an appropriate light detector. Microscope objectives of varying power, field diameter, and focal length are readily utilized as at least a portion of this optical train. The light detectors are optionally photodiodes, avalanche photodiodes, photomultiplier tubes, diode arrays, or in some cases, imaging systems, such as charged coupled devices (CCDs) and the like. In preferred aspects, photodiodes are utilized, at least in part, as the light detectors. The detection system is typically coupled to a computer (described in greater detail below), via an analog to digital or digital to analog converter, for transmitting detected light data to the computer for analysis, storage and data manipulation.

In the case of fluorescent materials such as labeled cells, the detector typically includes a light source which produces light at an appropriate wavelength for activating the fluorescent material, as well as optics for directing the light source through the detection window to the material contained in the channel or chamber. The light source can be any number of light sources that provides an appropriate wavelength, including lasers, laser diodes and LEDs. Other light sources are required for other detection systems. For example, broad band light sources are typically used in light scattering/transmissivity detection schemes, and the like. Typically, light selection parameters are well known to those of skill in the art.

The detector can exist as a separate unit, but is preferably integrated with a controller system, into a single instrument such as a benchtop research station. Integration of these functions into a single unit facilitates connection of these instruments with the computer (described below), by permitting the use of few or a single communication port(s) for transmitting information between the controller, the detector and the computer.

F. Computer

As noted above, either or both of the fluid direction system and/or the detection system are coupled to an appropriately programmed processor or computer which functions to instruct the operation of these instruments in accordance with preprogrammed or user input instructions, receive data and information from these instruments, and interpret, manipulate and report this information to the user. As such, the compute is typically appropriately coupled to one or both of these instruments (e.g., including an analog to digital or digital to analog converter as needed).

The computer typically includes appropriate software for receiving user instructions, either in the form of user input into a set parameter fields, e.g., in a GUI, or in the form of preprogrammed instructions, e.g., preprogrammed for a variety of different specific operations. The software then converts these instructions to appropriate language for instructing the operation of the fluid direction and transport controller to carry out the desired operation. For example, the software optionally directs the fluid direction system to alternately apply pressure to a sample reservoir and to a buffer well to create sample and buffer plugs, while simultaneously directing a the fluid direction system to apply pressure to a main channel to flow the sample and buffer plugs through the main channel. Any other movement necessary to assay, separate, or detect the sample is also optionally directed by the software instructions.

The computer then receives the data from the one or more sensors/detectors included within the system, and interprets the data, either provides it in a user understood format, or uses that data to initiate further controller instructions, in accordance with the programming, e.g., such as in monitoring and control of flow rates, temperatures, applied voltages, and the like.

The above integrated system components, e.g., fluid direction systems, detectors, and computers, and the like, are typically incorporated into a single unit that functions as an individual benchtop research station. Using the emulator devices, e.g., as described in the examples below, with an integrated system, e.g., the Agilent 2100 Bioanalyzer from Agilent Technologies (Palo Alto, Calif.), allows an assay to be designed, debugged, and optimized for high throughput scale up, e.g., using a sipper device.

IV. Example Systems

A. Steady State Emulator Device

An example steady state emulator device is illustrated, e.g., in FIG. 2, Panel A. Device 200 comprises two substantially identical, e.g., fluidically identical, and independent fluid channel circuits 202 and 204, each of which comprises a planar equivalent of a sipper device. For simplicity, only one circuit is described below. Capillary emulator channel 216 is fluidly coupled to reservoir 208. In an enzymatic assay, an enzyme is optionally introduced through reservoir 210 while substrate is optionally introduced through well 206. Fluid flow is typically driven by pressure sources, e.g., a single vacuum source coupled to reservoir 212. The hydrodynamic resistances for each channel, e.g., channels 214, 218, and 220, in the emulator device are substantially equivalent to those in the sipper device being emulated. As a consequence the fraction of total flow in main channel 220 delivered from capillary emulator channel 216, enzyme channel 218, and substrate channel 214 is equivalent to the total flow in a sipper device. In addition, the incubation time of enzyme and substrate in the emulator device for a given vacuum set point is substantially identical to the incubation time for the emulated sipper device.

B. Emulation of a Sipper Device in the Steady State Emulator Device

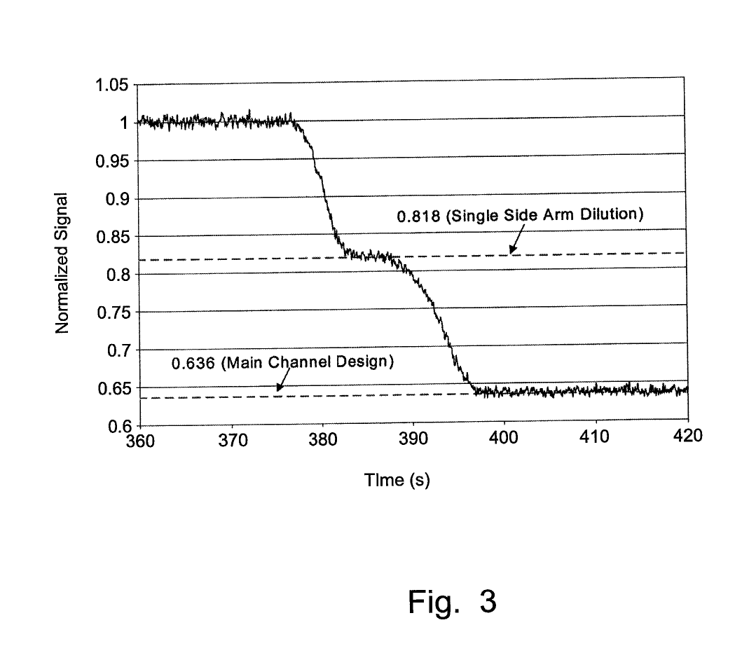

The device of FIG. 2 was used to simulate sipper device fluid flow. The fraction of total flow delivered from the side channels, e.g., channels 218 and 214, and capillary emulator channel 216 was measured experimentally and compared to a given design for a sipper device. For example, a given design is optionally a model based on a hydrodynamic analog to an electrical circuit. Hydrodynamic resistances are optionally computed based on channel geometry, e.g., width, depth, length, and the like. A fraction of flow supplied by a fluidic connection is then calculated based on resistances and connectivity. In addition, transit times and dilution ratios are optionally calculated based on resistances and connectivity. Using a device of FIG. 2, initially, dye was wicked into the entire fluidic circuit via capillary emulator channel 216. Next, enzyme reservoir 210 and substrate reservoir 206 were filled with buffer and the device was placed in a single vacuum port microscope station, e.g., with a single vacuum source fluidly coupled to main channel 220. The detection region for the microscope station was located on main reaction channel 220 in detection region 222. The washout of dye from each of the side channels, e.g., channels 214 and 218, was examined for each channel to determine their individual contributions to the total flow in the main channel. FIG. 3 shows a typical washout experiment in which the data has been normalized to the full-scale dye signal. The time delay between the enzyme and substrate channel is used to determine the flow fraction from each channel, with the washout test producing a characteristic stair step profile as shown in FIG. 3. Experimentally, the side channels were found to each deliver 18.2% of the total flow in the main channel, in excellent agreement with the model design.

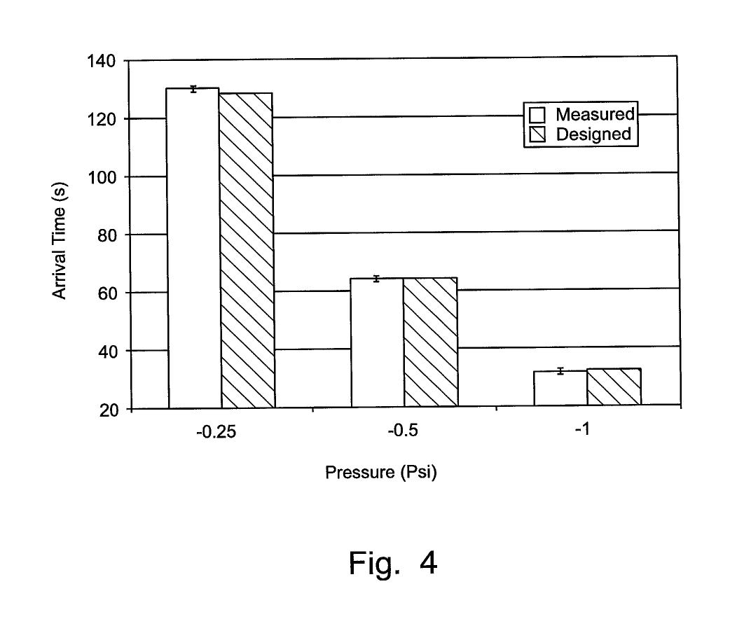

In addition to the side arm dilution, the enzyme and substrate incubation time was experimentally measured and compared to the model design. Initially, reservoir 208 was filled with dye and the remaining reservoirs were filled with buffer. The device was placed in the single vacuum port microscope station with the detection point located in detection region 222. The flow time from capillary emulator channel 216 to the detection point was measured by monitoring the arrival time of a flow perturbation introduced by a rapid voltage pulse between reservoirs 208 and 210. Velocimetry results compared to the model predictions are shown in FIG. 4 for three operating pressures: −0.25 psi, −0.5 psi, and −1 psi.

C. Dual-mode Injector Emulator Device

A dual-mode electrokinetic injector planar emulator of a sipper device is illustrated in FIG. 11. Channels that simulate a sipping capillary, e.g., channels fluidly coupled to reservoirs 1102, 1104, and 1110, form capillary emulator 1116. To perform an enzyme inhibition assay, an enzyme is flowed into main channel 1122 from reservoir 1106, e.g., via enzyme channel 1124. A substrate is optionally flowed into main channel 1122 from reservoir 1112, e.g., via substrate channel 1126. Fluid is typically driven through main channel 1122 towards detection region 1118, e.g., via a single vacuum source connected, e.g., to reservoir 1108. Inhibitor sample plugs are generated by electrokinetic pulses applied across cross-junction 1120 in capillary emulator 1116. For example, an inhibitor sample is optionally placed in reservoir 1104 and a buffer in reservoirs 1102 and 1110. The pulses are generated by switching the electrokinetic flow between two states: (1) inhibitor to waste, e.g., reservoir 1104 to reservoir 1102, and (2) buffer to waste, e.g., reservoir 1110 to reservoir 1102. The electrokinetic flow used to generate the pulses that produce the sample plugs does not alter the pressure driven flow downstream of capillary emulator 1116, e.g., the injector. In addition, emulator device 1100 is optionally fabricated to emulate sample dispersion incurred in the capillary of a typical sipper device. For example, the channel width, depth, and length of the straight section of capillary emulator 1116 downstream of the cross-junction, e.g., region 1130, are optionally chosen to produce the equivalent amount of dispersion as a corresponding sipper device capillary. In addition, sipper device flow rates and incubation times are optionally emulated to produce equivalent rates and times in the dual-mode emulator device.

Alternatively, a planar injector is optionally substantially pressure driven. For example, a pressure gradient is optionally used to load samples into the channel structure, e.g., by creating sample plugs by alternately applying pressure between the inhibitor and waste reservoirs and between the buffer and waste reservoirs.

D. Emulation of a Sipper Device Using a Dual-mode Emulator Device

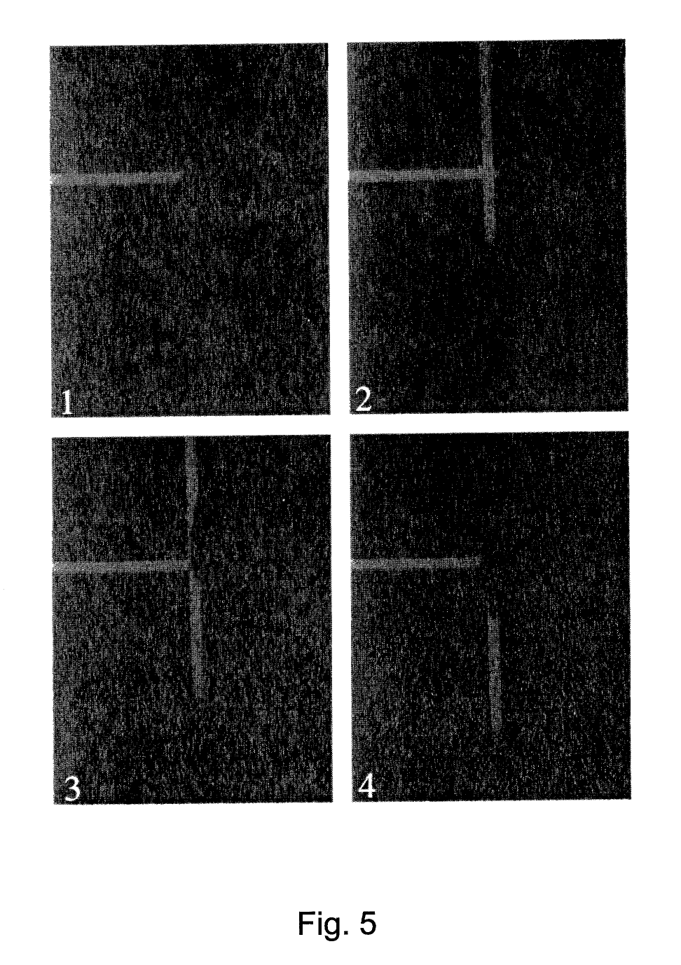

A dual-mode injector as shown in FIG. 11 was tested experimentally using a single vacuum port research microscope. A typical dye injection experiment is shown in FIG. 5. The field of view shown in FIG. 5 is zoomed in on cross-junction 1120 of the device shown in FIG. 11. This is where the dual mode injection takes place. Initially, the dual mode injector is in state (2) in which a potential difference exists between buffer reservoir 1110 and waste well 1102. This is shown in Panel A of FIG. 5. Next, the injector is switched to state (1) filling cross-junction 1120 with dye which is supplied to the bottom region of the junction by the steady state pressure driven flow, e.g., along channel region 1130 and main channel 1122. This is illustrated in Panel B of FIG. 5. Finally, the injector is switched back to state (2), creating a discrete plug of dye separated on either side with buffer. This is illustrated in panels C and D of FIG. 5.

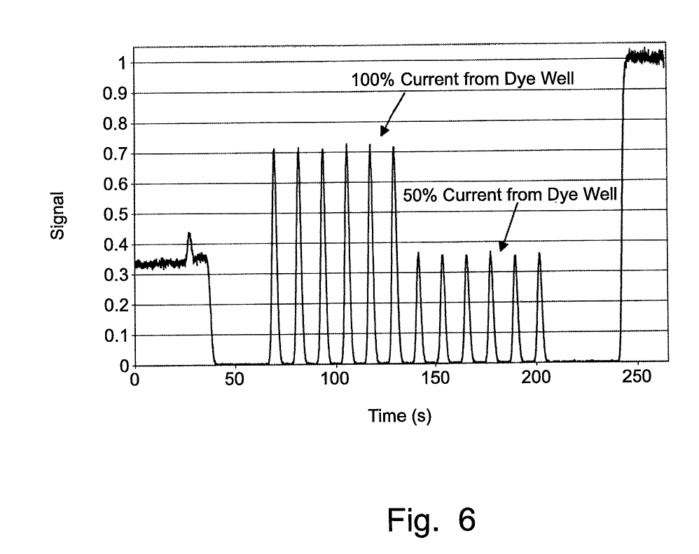

Typical dye injection data is shown in FIG. 6, in which the signal is normalized relative to steady dye flow from the capillary emulator, e.g., capillary emulator 1116. For example, the signal is normalized to a situation in which the injector is in state (1) as described above. At the start of the experiment, the injector is switched to flow buffer from capillary emulator 1116 for 30 seconds, followed by a series of 2 second dye injections with a 10 second buffer spacer, using an 8 mA switching current. The second set of peaks in FIG. 6 were performed at half concentration by dividing the switching current between the sample and the buffer wells in state (1), e.g., reservoirs 1104 and 1110. At the end of the experiment, the injector is switched to state (1), or 100% dye flow from capillary emulator 1116. A normalized signal of approximately 0.33 for t<40 seconds corresponds to approximately one-third flow from each of the injector arms or channels into the main channel, e.g., channel 1122, driven only by pressure. The rapid drop in baseline at approximately 40 seconds corresponds to transit time from the capillary emulator to the detector for the initial injector switch to state (2) at t=0.

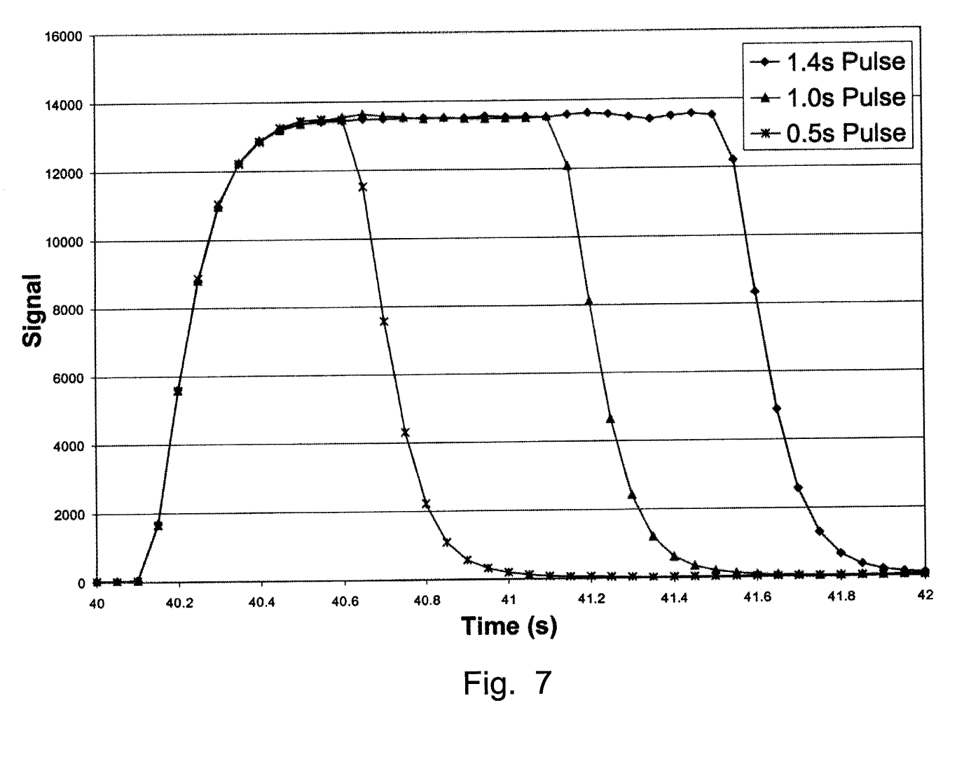

The resolution of the dual-mode injector was examined experimentally by placing the detector immediately downstream of the injector cross-junction, e g., cross-junction 1120, where the sample plugs are formed. FIG. 7 shows the signal obtained for 0.5 second, 1.0 second, and 1.4 second dye pulses, indicating that the dual mode injector is more than adequate for modeling typical sipper device injections.

The above experiments demonstrate the use of emulator devices, e.g., for fluorogenic assays. The devices are optionally used to aid in assay development processes for sipper formats. For example, enzyme and substrate concentrations are optionally optimized in a planar emulator device prior to performing high throughput assays, e.g., for determining kinetic parameters, on a sipper device. In addition, the ability to view the microfluidic network in a microscope station or platform allows the user to observe the reaction and debug other potential issues, e.g., chemical sticking to the channel walls. In addition, sip timing is also optimized, e.g., for inhibitor and buffer plugs. Furthermore, the ability to deliver discrete plugs of sample under pressure driven flow using a planar emulator device allows better understanding of dispersion characteristics of complex microfluidic channel geometries, including steps, bends, and funnels, e.g., prior to introducing such geometries into a high throughput sipper device.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above may be used in various combinations. All publications, patents, patent applications, or other documents cited in this application are incorporated by reference in their entirety for all purposes to the same extent as if each individual publication, patent, patent application, or other document were individually indicated to be incorporated by reference for all purposes.

What is claimed is:

1. A method of flowing fluid in a non-sipper microfluidic device, the method comprising: flowing fluid through the non-sipper microfluidic device to emulate a fluid flow profile in a microfluidic device comprising an external capillary, wherein the fluid flow profile results from flowing one or more sample from an external source into a microfluidic device.

2. The method of claim 1, wherein the non-sipper microfluidic device comprises a planar microfluidic device.

3. The method of claim 1, wherein the external source comprises a microwell plate.

4. The method of claim 1, wherein flowing fluid through the non-sipper microfluidic device comprises creating one or more sample plug and one or more buffer plug in the non-sipper microfluidic device, which one or more sample plug and one or more buffer plug emulate fluid flow from the external source into the microfluidic device via the external capillary.

5. The method of claim 4, wherein creating the one or more sample plug and the one or more buffer plug comprises:
(i) loading a sample from a first source into a channel of the non-sipper microfluidic device,
(ii) loading a buffer from a second source into the channel;
(iii) applying pressure to the sample in the channel, thereby creating the one or more sample plug and transporting the one or more sample plug through the channel; and,
(iv) applying pressure to the buffer in the channel, thereby creating the one or more buffer plug and transporting the one or more buffer plug through the channel.

6. The method of claim 5, comprising alternately performing step (i) and step (ii).

7. The method of claim 5, comprising repeating steps (i) and (ii).

8. The method of claim 5, comprising continuously performing step (iii) and step (iv).

9. The method of claim 5, comprising alternately performing step (i) and step (ii) while simultaneously performing step (iii) and step (iv).

10. The method of claim 5, step (iii) and step (iv) comprising simultaneously applying a first pressure to the sample and a second pressure to the buffer, wherein the first pressure and the second pressure are different.

11. The method of claim 5, wherein the first source and the second source comprise internal reservoirs.

12. The method of claim 5, comprising loading the sample from the first source into the channel of the non-sipper microfluidic device by applying a first electrokinetic gradient between the first source and a waste reservoir and loading the buffer from the second source into the channel by applying a second electrokinetic gradient between the second source and the waste reservoir.

13. The method of claim 12, wherein the waste reservoir comprises an internal reservoir.

14. The method of claim 12, comprising alternately applying the first electrokinetic gradient and the second electrokinetic gradient.

15. The method of claim 14, comprising alternately applying the first electrokinetic gradient and the second electrokinetic gradient and simultaneously applying pressure to the sample in the channel and to the buffer in the channel.

16. The method of claim 5, comprising loading the sample from the first source into the channel by applying pressure to the sample and loading the buffer from the second source into the channel by applying pressure to the buffer.

17. The method of claim 16, comprising alternately applying pressure to the sample and to the buffer.

18. The method of claim 17, comprising alternately applying pressure to the sample in the first source and to the buffer in the second source and concurrently applying pressure to the sample in the channel and to the buffer in the channel.

19. The method of claim 1, wherein flowing fluid through the non-sipper microfluidic device comprises
(i) flowing a sample from a first internal source into a non-sipper main channel via a capillary emulator channel;
(ii) flowing the sample through the non-sipper main channel; and,
(iii) flowing one or more reagent from at least a second internal source into the non-sipper main channel via a non-sipper side channel.

20. The method of claim 19, wherein the capillary emulator channel simulates the external capillary.

21. The method of claim 19, wherein the non-sipper main channel simulates a sipper main channel.

22. The method of claim 19, wherein the non-sipper side channel simulates a sipper side channel.

23. The method of claim 20, claim 21, or claim 22, wherein simulates comprises having substantially the same hydrodynamic resistance as an equivalent channel in the microfluidic device comprising the external capillary.

24. The method of claim 20, claim 21, or claim 22, wherein simulates comprises having substantially the same length, width, and depth as an equivalent channel in the microfluidic device comprising the external capillary.

25. The method of claim 20, claim 21, or claim 22, wherein simulates comprises flowing substantially the same amount of the one or more reagent or the sample as an equivalent channel in the microfluidic device comprising the external capillary.

26. An assay development device, which assay development device emulates a microfluidic sipper device, the assay development device comprising:
(i) a non-sipper microfluidic substrate comprising a plurality of microscale channels, the plurality of microscale channels comprising:
(a) a main channel; and,
(b) at least one capillary emulator fluidly coupled to the main channel; and,
(ii) at least a first fluid control element fluidly coupled to the main channel in the non-sipper microfluidic device.

27. The assay development device of claim 26, wherein the non-sipper microfluidic substrate comprises a planar microfluidic substrate.

28. The assay development device of claim 26, which main channel emulates a sipper device main channel.

29. The assay development device of claim 28, wherein the main channel comprises a first hydrodynamic resistance, a length, a width, a depth, or a flow characteristic, which hydrodynamic resistance, length, width, depth, or flow characteristic is substantially equal to the sipper device main channel.

30. The assay development device of claim 26, wherein the capillary emulator comprises a microscale channel, which microscale channel comprises a hydrodynamic resistance, a length, a width, a depth, or a flow characteristic, which hydrodynamic resistance, length, width, depth, or flow characteristic is substantially equal to a sipper capillary in the microfluidic sipper device.

31. The assay development device of claim 26, further comprising an electrokinetic controller fluidly coupled to the capillary emulator, which capillary emulator comprises:
a waste reservoir, which waste reservoir is fluidly coupled to the main channel;
a sample well fluidly coupled to the waste reservoir and to the main channel;
a buffer well fluidly coupled to the waste reservoir and to the main channel;
wherein the first fluid control element applies a pressure differential between the waste reservoir and the pressure source, and the electrokinetic controller alternately applies an electrokinetic gradient between the sample well and the waste reservoir and between the buffer well and the waste reservoir.

32. The assay development device of claim 26, wherein the capillary emulator comprises:
a sample well fluidly coupled to the main channel; and,
a buffer well fluidly coupled to the main channel;
wherein the first fluid control element
applies a first pressure to a sample in the sample well, thereby flowing the sample into the main channel;
applies a second pressure to a buffer in the buffer well, thereby flowing the buffer into the main channel; and,
applies a third pressure to the sample in the main channel or to the buffer in the main channel.

33. The assay development device of claim 32, wherein the fluid control element alternates between applying the first pressure and applying the second pressure and concurrently applies the third pressure.

34. The assay development device of claim 26, the first fluid control element comprising a pressure source or an electrokinetic controller.

35. An assay development device, the device comprising:
(i) a non-sipper microfluidic device comprising a plurality of microscale channels, the plurality of microscale channels comprising:
(a) a main channel;
(b) a first reagent well fluidly coupled to the main channel;
(c) a second reagent well fluidly coupled to the main channel; and,
(d) a waste reservoir fluidly coupled to the main channel, the first reagent well, and the second reagent well; and,
(ii) a fluid control system, which fluid control system comprises:
(a) a pressure source fluidly coupled to the main channel; and,
(b) an electrokinetic controller operably coupled to the main channel;
wherein the fluid control system applies a pressure differential between the waste reservoir and the pressure source, and alternately applies an electrokinetic gradient between the first reagent well and the waste reservoir and between the second reagent well and the waste reservoir.

36. A method of fabricating an assay development device, the method comprising:
(i) providing a non-sipper microfluidic substrate; and,
(ii) fabricating two or more channels within the non-sipper microfluidic substrate, the two or more channels emulating an external capillary and a main channel of a microfluidic sipper device.

37. The method of claim 36, the non-sipper microfluidic substrate comprising a planar substrate.

38. The method of claim 36, wherein emulating the external capillary and the main channel of the microfluidic sipper device comprises having one or more of: substantially the same hydrodynamic resistance, substantially the same width, substantially the same depth, substantially the same length, or substantially the same flow characteristics as the external capillary or the main channel of the microfluidic sipper device.

39. The method of claim 36, the two or more channels comprising two or more of: a capillary emulator, a main channel, a side channel, or a reservoir.

40. The method of claim 38, wherein having substantially the same flow characteristics as the external capillary or the main channel in the microfluidic sipper device comprises providing substantially the same amount of fluid flow in substantially the same amount of time.
